(19) United States
(12) Patent Application Publication
(10) Pub. No.: US 2015/0071941 A1
(43) Pub. Date: Mar. 12, 2015

(54) TREATMENT OF ISCHEMIC RETINOPATHIES

(71) Applicants: THE JOHNS HOPKINS UNIVERSITY, Baltimore, MD (US); UNIVERSITY OF MARYLAND, BALTIMORE, Baltimore, MD (US)

(72) Inventors: Akrit Sodhi, Cockeysville, MD (US); Silvia Montaner, Cockeysville, MD (US)

(21) Appl. No.: 14/394,152

(22) PCT Filed: Apr. 15, 2013
(86) PCT No.: PCT/US13/36561

§ 371 Date: Oct. 13, 2014

Related U.S. Application Data
(60) Provisional application No. 61/623,696, filed on Apr. 13, 2012.

(51) Int. Cl. C07K 16/18; A61K 39/395

(52) U.S. Cl. CPC C07K 16/18; A61K 39/3955; A61K 2039/505
USPC 424/158.1; 424/130.1

(57) ABSTRACT

The present invention relates to the field of ischemic retinopathies. More specifically, the present invention provides methods and compositions useful for treating ischemic retinopathies including diabetic macular edema (DME). The present invention also provides methods for treating ischemic retinopathy comprising the step of administering to a subject diagnosed with ischemic retinopathy an effective amount of an ANGPTL4 antagonist.

### CROSS-REFERENCE TO RELATED APPLICATIONS

This application claims the benefit of U.S. Provisional Application No. 61/623,696, filed Apr. 13, 2012; which is incorporated herein by reference in its entirety.

### STATEMENT OF GOVERNMENTAL INTEREST

This invention was made with U.S. government support under grant no. K08-EY021189. The U.S. government has certain rights in the invention.

### FIELD OF THE INVENTION

The present invention relates to the field of ischemic retinopathies. More specifically, the present invention provides methods and compositions useful for treating ischemic retinopathies.

### INCORPORATION-BY-REFERENCE OF MATERIAL SUBMITTED ELECTRONICALLY

This application contains a sequence listing. It has been submitted electronically via EFS-Web as an ASCII text file entitled “P11983-02_Sequence_Listing.” The sequence listing is 3,053 bytes in size, and was created on Apr. 15, 2013. It is hereby incorporated by reference in its entirety.

### BACKGROUND OF THE INVENTION

Ischemic retinopathies include a diverse group of retinal diseases in which immature retinal vasculature (e.g., retinopathy of prematurity, incontinentia pigmenti) or damage to mature retinal vessels (e.g., diabetic retinopathy, retinal vein occlusion, sickle cell retinopathy) leads to retinal ischemia (1). While diverse (and poorly understood) etiologies may lead to insufficient perfusion of the retina, all lead to a common sequela: the formation of abnormal, leaky blood vessels. This can manifest clinically with the accumulation of fluid in the inner retina (i.e., macular edema) and often a profound loss of vision (2). Indeed, macular edema in patients with ischemia-induced retinopathies remains the leading cause of vision loss in the working-age population in the developed world (3).

The concept that ischemic retinopathies are driven by ischemia-induced angiogenic factor(s) was proposed over half a century ago (4). A single transcriptional activator, hypoxia-inducible factor (HIF)-1, has recently emerged as the master regulator of these angiogenic mediators. HIF-1 is a heterodimeric protein composed of an exquisitely oxygen-sensitive a subunit α and a ubiquitous β subunit. Under hypoxic conditions, degradation of the oxygen-sensitive HIF-1α subunit is reduced while its transcriptional activity is enhanced (5-7). The resulting increased amount of active HIF-1α protein localizes to the nucleus and binds to HIF-1β forming a heterodimer (HIF-1) that is capable of binding to the DNA of specific (hypoxia-inducible) genes, and inducing broad changes in gene expression that mediate acclimation of cells, tissues, and the organism to conditions of low oxygen tension (8).

Although several HIF-1-dependent factors have been previously reported to stimulate retinal neovascularization, surprisingly few have been proven to play a significant role in the promotion of vascular permeability and macular edema. Arguably the most critical of the HIF-1-dependent secreted factors elaborated by hypoxic cells in ischemic retinopathies is vascular endothelial growth factor (VEGF) (9). VEGF, originally identified as vascular permeability factor, is a potent inducer of vessel permeability and macular edema (10). The recent development of specific monoclonal antibodies directed against VEGF has revolutionized the treatment of diabetic macular edema (DME). Results from recent clinical trials using anti-VEGF therapies for DME have demonstrated remarkable results, not only maintaining but also improving visual acuity (11). Nonetheless, though anti-VEGF treatment has shown better outcomes than alternative treatments, only a minority of patients with DME treated monthly with intravitreal injections of anti-VEGF therapies achieve a significant improvement in visual acuity (i.e., a gain of at least 15 letters—or three lines—on the ETDRS vision chart) following treatment (12). Moreover, some of these patients suffer from persistent or worsening edema and/or vision loss despite treatment. These observations suggest that other HIF-1-dependent genes may contribute to the pathogenesis of macular edema in these patients.

### SUMMARY OF THE INVENTION

The present invention is based, at least in part, on the discovery that inhibiting angiopoeitin-like 4 (ANGPTL 4) can be a useful therapy for treating ischemic retinopathies including diabetic macular edema (DME), vein occlusions and retinal and choroidal neovascular diseases including proliferative diabetic retinopathy (PDR) and neovascular age-related macular degeneration (AMD), respectively.

The anticipated rise in the global prevalence of diabetes will undoubtedly result in a concurrent increase in the number of patients with vision impairment from DME, already among the most common causes of severe vision loss in the developed world. Clinical trials assessing the efficacy of therapies targeting vascular endothelial growth factor (VEGF) have demonstrated a major improvement in vision in a minority of patients with DME. This underscores the need for a better understanding of DME pathogenesis and the identification of novel factors contributing to this disease. Here, we provide evidence implicating hypoxic Müller glial cells as the driving force behind vascular permeability in DME by stabilizing hypoxia-inducible factor (HIF)-1α. Increased HIF-1α, in turn, promotes the transcription of cytokines (e.g., VEGF) secreted by Müller cells. Blocking HIF-1α translation in Müller cells with digoxin inhibits their promotion of endothelial cell permeability in vitro and retinal edema in vivo. Interestingly, Müller cells strictly require HIF—but not VEGF—to promote vascular permeability, implicating additional HIF-dependent factors in DME pathogenesis. Using gene expression analysis, we identify angiopoietin-like 4 (ANGPTL4) as a novel cytokine potently upregulated by HIF-1 in hypoxic Müller cells in vitro and in the ischemic inner retina in vivo. ANGPTL4 is critical and sufficient for the promotion of vessel permeability by hypoxic Müller cells. Immunohistochemical analysis of retinal tissue from patients with DME demonstrates that HIF-1α and ANGPTL4 co-localize to ischemic Müller cells. We conclude that HIF-1α and ANGPTL4 are essential targets for the treatment of DME.

Accordingly, in one aspect, the present invention provides methods and composition useful for treating ischemic retinopathies. In one embodiment, a method for treating diabetic macular edema (DME) comprises the step of administering to a subject diagnosed with DME an effective mount of an angiopoietin-like 4 protein (ANGPTL4) antagonist, wherein the effective amount treats DME in the subject. In a specific embodiment, the ANGPTL4 antagonist is an anti-ANGPTL4 antibody. In another embodiment, the method further comprises the step of administering an effective amount of a vascular endothelial growth factor (VEGF) antagonist, wherein the combined effective amount treats DME in the subject. In a specific embodiment, the VEGF antagonist is an anti-VEGF antibody. In another embodiment, the administration of the ANGPTL4 antagonist and the VEGF antagonist step is performed concurrently. In yet another embodiment, the ANGPTL4 antagonist and the VEGF antagonist are administered as a combination composition. In particular embodiments, the antagonist is administered intraocularly.

In another embodiment of the present invention, a method for treating DME comprises the step of administering to a subject diagnosed with DME an effective amount of an ANGPTL4 antagonist and an effective amount of a VEGF antagonist, wherein the combined effective amounts treat DME in the subject. In a specific embodiment, the ANGPTL4 antagonist is an anti-ANGPTL4 antibody. In another specific embodiment, the VEGF antagonist is an anti-VEGF antibody. In a further embodiment, the administration of the ANGPTL4 antagonist and the VEGF antagonist step is performed concurrently. In an alternative embodiment, the ANGPTL4 antagonist and the VEGF antagonist are administered as a combination composition.

The present invention also provides methods for treating ischemic retinopathy comprising the step of administering to a subject diagnosed with ischemic retinopathy an effective amount of an ANGPTL4 antagonist. In certain embodiments, the ANGPTL4 antagonist is a small molecule, an antibody, polypeptide, a polynucleotide, an aptamer, or an siRNA. In particular embodiments, the ischemic retinopathy is DME or neovascular age-related macular degeneration (AMD). In other embodiments, the ischemic retinopathy is retinopathy of prematurity, incontinentia pigmenti, diabetic retinopathy, retinal vein occlusion, and sickle cell retinopathy.

In one embodiment, a method for treating DME comprises the step of intraocularly administering to a subject diagnosed with DME an effective amount of an ANGPTL4 antibody. In another embodiment, a method for treating AMD comprises the step of intraocularly administering to a subject diagnosed with neovascular AMD an effective amount of an ANGPTL4 antibody. The present invention also provides a method for treating DME comprising the step of intraocularly administering to a subject diagnosed with DME an effective amount of an ANGPTL4 antibody and an effective amount of a VEGF antibody.

In yet another embodiment, a method for inhibiting ANGPTL4 in a patient diagnosed with ischemic retinopathy comprises the step of administering an effective amount of an ANGPTL4 antagonist. In certain embodiments, the ischemic retinopathy is DME, proliferative diabetic retinopathy (PDR) or neovascular AMD. In other embodiments, the ischemic retinopathy is one or more conditions selected from the group consisting of retinopathy of prematurity, incontinentia pigmenti, diabetic retinopathy, retinal vein occlusion, sickle cell retinopathy, neovascular glaucoma, corneal neovascularization, pterygia, and pinguecula, vein occlusions including diabetic macular edema (DME), vein occlusions and retinal and choroidal neovascular diseases.

### BRIEF DESCRIPTION OF THE FIGURES

FIG. 1: HIF and GFAP expression co-localize to injured Müller cells in the Oxygen-Induced Retinopathy (OIR) Model. Representative images from immunofluorescent analysis demonstrating HIF-1α protein levels are increased (purple arrow) in P13 OIR mice (24 hours post hyperoxia) in the posterior (hypoxic) inner retina within the inner nuclear layer and localizes to areas of increased expression of GFAP (green arrow) in injured Müller glial cells. Accumulation of HIF-1α protein is inhibited by daily intraperitoneal injections of digoxin (2 mg/kg), to levels similar to those in P13 control mice. n=6 animals in each group.

FIG. 2: HIF-1α protein accumulation and VEGF expression in cultured hypoxic Müller cells and in patients with diabetic macular edema. (A) Primary murine Müller cells isolated from mouse retinas at age P0-P5 express key Müller cell markers (vimentin, GFAP, and CRALBP). (B to D) Exposure of primary mouse Müller cells to hypoxia (8 hours) induces HIF-1α protein stability (B) and nuclear localization (C) and results in increased Vegf mRNA (D). (E to G) Exposure of immortalized human Müller (MIO-M1) cells to hypoxia (8 hours, or for indicated time) induces HIF-1α protein stability (E) and nuclear localization (F) and results in increase VEGF mRNA and protein (G). (H) Representative images from immunohistochemical analysis of DME eyes from patients with known DME (n=5 eyes) reveals the presence of activated (vimentin and GFAP-expressing) Müller cells in the ischemic (posterior) retina, but not in the peripheral retina (vimentin-expressing only). Similar to GFAP, HIF-1α and VEGF protein was also detected in cells in the inner retina in the posterior but not peripheral retina. Student's t test, * p<0.05; ** p<0.01.

FIG. 3: Inhibition of HIF-1 translation with digoxin blocks vascular permeability in the OIR Model. (A and B) Representative images from H & E and immunohistochemical analysis of P13 OIR mice (24 hours post hyperoxia) demonstrate an increase in inner retinal cysts (A; see inset, black arrows) and extravasation of albumin (B, immunohistochemistry; blue arrows) compared to control animals. Treatment of OIR mice with daily intraperitoneal injections with the HIF inhibitor, digoxin (2 mg/kg), inhibits the increase in inner retinal cysts and extravasated albumin. (C) Representative images from immunofluorescent analysis demonstrating extravascular albumin (green arrows) co-localizing with the inner retinal capillaries (CD31; red arrows) in the P13 OIR mouse, but not in the control mouse, nor in P13 OIR mouse treated with daily intraperitoneal injections of digoxin. n=6 animals in each group.

FIG. 4: HIF-1-dependent promotion of endothelial cell permeability by hypoxic Müller cells. (A) Exposure of a monolayer of human dermal microvascular endothelial cells (HMVECs) to conditioned media from MIO-M1 cells exposed to hypoxia (8 hours) promotes endothelial cell permeability (as measured by passage of FITC-Dextran through the monolayer). (B and C) Pre-treatment with digoxin (100 nM; 2 hours prior to exposure to hypoxia) inhibits hypoxia-induced HIF-1α protein stabilization (B), VEGF RNA transcription and VEGF secretion (C; 8 hours hypoxia) in MIO-M1 cells. (D) Pre-treatment with 100 nM digoxin (2 hours prior to 8 hour hypoxia exposure), inhibits the promotion of endothelial cell permeability by hypoxic MIO-M1 cells. (E) Pre-treatment (2 hours prior hypoxia) with the VEGF receptor (KDR) inhibitor (SU1498; 5 μM) only partially blocks the promotion of endothelial cell permeability by MIO-M1 cells exposed to 8 hours of hypoxia but abolishes the promotion of endothelial cell permeability by 100 ng of recombinant human VEGF. Student's t-test, * p<0.05; ** p<0.01.

FIG. 5: HIF-1-dependent upregulation of ANGPTL4 by hypoxic Müller cells in vitro. (A to C) ANGPTL4 RNA (A) and protein (B and C; arrow points to top band in WB that corresponds to ANGPTL4) increases with exposure of MIO-M1 cells to hypoxia, but is inhibited by pretreatment with the HIF inhibitor, digoxin (100 ng, 2 hours prior hypoxia). (D and E) Exposure of primary murine Müller cells to hypoxia (8 hours) results in an increase in HIF-1α protein accumulation (D) and a corresponding increase in Angptl4 RNA (E). (F-J) Infection of MIO-M1 cells with adenovirus expressing a constitutively-active deletion mutant of HIF-1α (Ad-CA5) results in accumulation of the stable CA5-HIF-1α (F) and an increase in VEGF and Angptl4 mRNA (G and I) and protein (H and J) under normoxic (20% oxygen) conditions. Student's t-test, * p<0.05; ** p<0.01.

FIG. 6: HIF-1-dependent upregulation of ANGPTL4 in ischemic inner retina in vivo. (A) RT-PCR of Angptl4 and Vegf RNA from the neurosensory retina of OIR animals at P12 to P15 normalized to cyclophilin A mRNA, and reported as fold induction compared to P12. (B) Representative images from H and E or immunohistochemical analysis of VEGF and ANGPTL in the retina of P13 OIR animals demonstrating expression in the inner posterior retina (see inset) Immunohistochemical analysis of ANGPTL4 levels in age-matched non-OIR pups was low. No primary antibody was used for the negative control. (C) Representative Western Blot of HIF-1α protein accumulation in OIR model with (+) or without (−) daily intraperitoneal injection of digoxin. (D) RT-PCR of Angptl4 and Vegf mRNA from the neurosensory retina of OIR animals at P12 to P14 with (+dig) or without daily intraperitoneal injection of digoxin, normalized to cyclophilin A mRNA, and reported as fold induction compared to P12. (E) Schematic demonstrating intravitreal injection of Adenovirus expressing LacZ control (Ad-LacZ) or a constitutively-active mutant of HIF-1α (Ad-CA5). (F) Western Blot demonstrating accumulation of stable HIF-1α in the neurosensory retina in animals infected with Ad-CM. (G) RT-PCR of Angptl4 mRNA from the neurosensory retina of animals infected with Ad-LacZ (LacZ) or Ad-CA5 (HIF) normalized to cyclophilin A mRNA, and reported as fold induction compared to uninfected eyes. n=6 animals in each group. Student's t test, * p<0.05.

FIG. 7: ANGPTL4 promotes endothelial cell permeability in vitro and vascular permeability in the inner retina in vivo. (A) Exposure of a monolayer of HMVECs to recombinant human ANGPTL4 (2.5 or 5 μg) or to VEGF (100 ng) promotes endothelial cell permeability (as measured by passage of FITC-Dextran through the monolayer). (B) Modified Miles assay was used to measure vascular permeability in the mouse ear following intradermal injection with 20 μl of PBS, ANGPTL4 (0.2 μg) or VEGF (0.2 μg). Representative photos demonstrating leakage of dye at injection site minutes after injection. Quantitation of Evans blue dye using spectrophotometry (610 nm) demonstrates a marked increase in dye in ears injected with ANGPTL4 compared to PBS (similar to VEGF). n=3 animals in each group. (C) RNAi targeting ANGPTL4 blocks ANGPTL4 but not VEGF mRNA expression and protein secretion in transfected MIO-M1 cells, and inhibits the promotion of endothelial cell permeability by conditioned media from MIO-M1 cells exposed to hypoxia for 8 hours (C). (D) Representative H & E stained sections following intravitreal injection with 200 ng recombinant murine ANGPTL4 (rmANGPTL4) into the mouse eye, demonstrating increased inner retinal cysts (see inset, black arrows) compared to control (PBS-treated) eyes. (E) Representative images from immunohistochemical analysis demonstrates extravasation of albumin (blue arrows) in mouse eyes following intravitreal injection of 200 ng rmANGPTL4 compared to control animals. (F) Representative images from immunofluorescent analysis demonstrating albumin (green arrows) extravasation co-localizing with the inner retinal capillaries (CD31; red arrows) in mouse eyes following intravitreal injection of 200 ng rmANGPTL4 compared to control animals. n=6 animals in each group. Student's t test, * p<0.05. ***p<0.001.

FIG. 8: ANGPTL4 specifically expressed in the ischemic retina of patients with diabetic macular edema. Representative H & E staining and immunohistochemical analysis of eyes from patients with DME (n=5 eyes) reveals the presence of ANGPTL4 in the ischemic (posterior) retina, but not in the peripheral retina, similar to the expression of VEGF protein.

FIG. 9: Hypoxia in OIR Model. (A) Representative images from immunohistochemical analysis of CD31 (vascular endothelial marker) reveals vaso-obliteration of the posterior retinal vasculature during the hyperoxia phase (P7-P12) of the OIR model resulting in retinal hypoxia (HypoxyProbe) in the posterior—but not the peripheral—retina. (B) Retinal ischemia (HypoxyProbe) occurs in the inner retina and persists for several days. n=6 animals in each group.

FIG. 10: Injured (GFAP-expressing) Müller cells in posterior hypoxic inner retina in OIR model localize to cells expressing HIF-1α. (A) High magnification view of P13 OIR posterior retina from FIG. 1 demonstrating a close correlation between GFAP-expressing injured Müller cells and HIF-1α expression in the ischemic inner retina. (B) Representative images from immunohistochemical analysis of vimentin-expressing Müller glial cells observed in the posterior and peripheral inner retina in OIR mice. Increased expression of GFAP in injured Müller glial cells is seen only in the posterior (hypoxic) inner retina with few inner retinal vessels (CD31) 24 to 48 hours after OIR mice are returned to normoxia (relative hypoxia). n=6 animals in each group.

FIG. 11: Expression of Müller cell markers by isolated and cultured primary murine Müller cells. Western blot comparing expression of three Müller cell markers, CRAL BP (expressed by Müller and RPE cells), vimentin (expressed by Müller but not RPE cells), and GAPDH (expressed by Müller cells and astrocytes, but not RPE cells) in immortalized RPE (ARPE-19) and Müller (M10-M1) cell lines compared to late passage (8 passages) Müller cells isolated from the neurosensory retina of new born pups.

FIG. 12: Quantitation of vascular permeability in the inner retina of OIR mice. Vascular permeability in the inner retina in OIR mice (+/−-digoxin treatment) demonstrating the percentage of CD31-labled inner retina vessels in the posterior retina that had visible adjacent extravascular albumin (detected by immunofluorescence). n=3 animals in each group. * p<0.05; ** p<0.01.

FIG. 13: Inhibition of HIF-1 by rapamycin or by RNAi against HIF-1β inhibits the induction ANGPTL4 mRNA expression and protein secretion in Müller cells exposed to hypoxia. (A and B) Inhibition of HIF-1α expression by pre-treatment of MIO-M1 cells with 300 nM rapamycin two hours prior to exposure to hypoxia (for 8 hours) results in decreased HIF-1 alpha protein accumulation (A) and decreased induction of VEGF and Angptl4 mRNA and protein (B) under hypoxic conditions. (C and D) Similarly, modest inhibition of the HIF-1α (and HIF-2α) binding partner, HIF-1β, using RNAi (C) results in a decrease in the induction of VEGF and Angptl4 mRNA and protein (D) under hypoxic conditions. Of note, more potent inhibition of HIF-1β resulted in cell death upon exposure of cells to hypoxia.

FIG. 14: VEGF and vascular permeability in ischemic retinopathies. (A) Representative Hematoxylin and eosin (H & E) stained images of an intravitreal injection with 100 ng recombinant murine VEGF (rmVEGF) into the mouse eye demonstrating increased inner retinal cysts (see inset, black arrows) compared to control (PBS-treated) animals. (B) Representative images from immunohistochemical analysis demonstrates extravasation of albumin (blue arrows) in mouse eyes following intravitreal injection with 100 ng rmVEGF compared to control animals. (C) Representative images from immunofluorescence analysis demonstrating albumin (green arrows) extravasation co-localizing with the inner retinal capillaries (CD31; red arrows) in mouse eyes following intravitreal injection with 100 ng rmVEGF compared to control animals. n=6 animals in each group.

FIG. 15: Quantitation of vascular permeability in the inner retina of adult mice following treatment with an intravitreal injection of ANGPTL4. Vascular permeability in the inner retina in adult mice treated with an intravitreal injection of 1 μl of PBS (control) or ANGPTL4 (200 ng/μl) demonstrating the percentage of CD31-labled inner retina vessels in the posterior retina that had visible adjacent extravascular albumin (detected by immunofluorescence). VEGF (200 ng/μl) was used as a positive control. n=3 animals in each group. * p<0.05; ** p<0.01.

FIG. 16: ANGPTL4 and VEGF not expressed in the posterior retina of patients without diabetic eye disease. Representative H & E staining and immunohistochemical analysis of eyes from age-matched control patients without diabetes (n=5 eyes) reveals the absence of ANGPTL4 in the perfused posterior retina. VEGF (and to a much lesser extent, HIF-1α) was detected in control patients.

FIG. 17: HIF dependent upregulation of ANGPTL4 in ARPE19 cells.

FIG. 18: HIF dependent upregulation of ANGPTL4 in human ES derived RPE cells.

FIG. 19: HIF-dependent upregulation of ANGPTL4 in primary murine RPE Cells.

FIG. 20: Transient Hypoxia in laser-induced CNV mouse model.

FIG. 21: HIF, VEGF, and ANGPTL4 upregulation in laser-induced CNV mouse model.

FIG. 22: Upregulation of ANGPLT4 mRNA in subretinal lipid hydroperoxide CNV rat model.

FIG. 23: ANGPTL4 sufficient to promote NV in vitro and in vivo.

FIG. 24: ANPTL4 levels increased in patients with CNV.

FIG. 25A-D: Hypoxic upregulation of HIF necessary to promote retinal NV in vivo.

FIG. 26A-D: ANGPTL4 increase in patients with PDR.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

Vision loss from ischemic retinopathies (e.g., diabetic retinopathy) most commonly results from the accumulation of fluid in the inner retina (i.e., macular edema) (2). Macular edema is caused by an ischemia-induced hyper-permeability state, which is regulated, in part, by the cytokine vascular endothelial growth factor (VEGF). The recent development of therapies targeting VEGF has revolutionized the treatment of diabetic macular edema (DME). However, only a minority of patients with DME treated monthly with anti-VEGF therapies achieve a significant improvement in visual acuity following treatment, suggesting that other genes may contribute to the pathogenesis of macular edema.

A single transcriptional activator, hypoxia-inducible factor (HIF)-1, has emerged as the master regulator of VEGF and other ischemia-induced mediators (3). To determine the relative contribution of HIF-1 (and HIF-1-dependent factor(s) that participate with VEGF) to the promotion of vascular permeability (and, in turn, macular edema) in DME, we examined the regulation of HIF-1-dependent genes in the oxygen-induced retinopathy (OIR) model for ischemic retinal disease (4). We provide evidence implicating Müller glial cells as a key inner retinal cell in which hypoxic stabilization of the oxygen-sensitive α subunit of HIF-1 (HIF-1α) is increased. We further observed that increased HIF-1α in Müller cells, in turn, promotes the transcription and secretion of cytokines (e.g., VEGF) that promote vascular permeability. By blocking HIF-1α translation with the pharmacological inhibitor digoxin, we inhibited the promotion of endothelial cell permeability by Müller cells in vitro and the increase in vascular permeability and retina edema in the ischemic inner retina in vivo. Interestingly, we provide evidence suggesting that suggests that Müller cells strictly required HIF—but not VEGF—to promote vascular permeability. This implicated additional HIF-dependent factors in the pathogenesis of DME.

In this regard, several HIF-1-dependent factors (in addition to VEGF) have been reported to stimulate angiogenesis; however, surprisingly few have been proven to play a significant role in pathogenesis of DME. We therefore used gene expression analysis to identify HIF-dependent genes upregulated by hypoxic Müller cells that may participate (with VEGF) in the promotion of vascular permeability and macular edema in diabetic eye disease. We identified angiopoietin-like 4 (ANGPTL4) as a cytokine upregulated in hypoxic Müller cells in vitro and in the ischemic inner retina in vivo. Inhibition of HIF-1α translation with digoxin blocked ANGPTL4 transcription and secretion by Müller cells in culture and its expression in the ischemic inner retina of mice. Conversely, forced HIF-1α expression was sufficient to promote an increase in ANGPTL4 transcription and secretion in Müller cells in vitro and in the retinas of mice in vivo. Inhibition of ANGPTL4 transcription with RNA interference (RNAi) significantly reduced the endothelial cell permeability promoted by hypoxic Müller cells while intravitreal injections with recombinant murine ANGPTL4 was sufficient to promote vascular permeability and retinal edema in mice Immunohistochemical analysis of retina tissue demonstrated that HIF-1α and ANGPTL4 localize to the ischemic inner retina, and, in particular, to activated (GFAP-expressing) Müller cells in patients with known DME.

### I. Definitions

The term “ANGPTL4 or “Angptl4” refers to angiopoietin-like 4 polypeptide or protein, along with naturally occurring allelic, secreted, and processed forms thereof. For example, human ANGPTL4 is a 406 amino acid protein, while murine ANGPTL4 is a 410 amino acid protein. The term “ANGPTL4” is also used to refer to fragments (e.g., subsequences, truncated forms, etc.) of the polypeptide comprising, e.g., N-terminal fragment, Coiled-coil domain, C-terminal fragment, fibrinogen-like domain of the ANGPTL4 protein. Reference to any such forms of ANGPTL4 can also be identified in the application, e.g., by “ANGPTL4 (23-406),” “ANGPTL4 (184-406),” “ANGPTL4 (23-183),” “ANGPTL4 (23-410),” “mANGPTL4 (184-410),” etc., where m indicates murine sequence. The amino acid position for a fragment native ANGPTL4 are numbered as indicated in the native ANGPTL4 sequence. For example, amino acid position 22(Ser) in a fragment ANGPTL4 is also position 22(Ser) in native human ANGPTL4. Generally, the fragment native ANGPTL4 has biological activity.

The term “ANGPTL4 modulator” refers to a molecule that can activate, e.g., an agonist, ANGPTL4 or its expression, or that can inhibit, e.g., an antagonist (or inhibitor), the activity of ANGPTL4 or its expression. ANGPTL4 agonists include antibodies and active fragments. An ANGPTL4 antagonist refers to a molecule capable of neutralizing, blocking, inhibiting, abrogating, reducing or interfering with ANGPTL4 activities, e.g., cell proliferation or growth, migration, adhesion or metabolic, e.g., lipid, modulation, or its expression including its binding to an ANGPTL4 receptor. ANGPTL4 antagonists include, e.g., anti-ANGPTL4 antibodies and antigen-binding fragments thereof, receptor molecules and derivatives which bind specifically to ANGPTL4 thereby sequestering its binding to one or more receptors, anti-ANGPTL4 receptor antibodies and ANGPTL4 receptor antagonists such as small molecule inhibitors of the receptor. Other ANGPTL4 antagonists also include antagonist variants of ANGPTL4, antisense molecules (e.g., ANGPTL4-SiRNA), RNA aptamers, and ribozymes against ANGPTL4 or its receptor. In certain embodiments, antagonist ANGPTL4 antibodies are antibodies that inhibit or reduce the activity of ANGPTL4 by binding to a specific subsequence or region of ANGPTL4, e.g., N-terminal fragment, Coiled-coil domain, C-terminal fragment, fibrinogen-like domain, and the like.

The term “Anti-ANGPTL4 antibody” is an antibody that binds to ANGPTL4 with sufficient affinity and specificity. The anti-ANGPTL4 antibody of the invention can be used as a therapeutic agent in targeting and interfering with diseases or conditions wherein ANGPTL4 activity is involved. Generally, an anti-ANGPTL4 antibody will usually not bind to other ANGPTL4 homologues, e.g., ANGPTL3. See U.S. Pat. No. 8,354,103; and U.S. Patent Applications Publication No. 20120171217 and No. 2011-0311524.

The terms “VEGF” and “VEGF-A” are used interchangeably to refer to the 165-amino acid vascular endothelial cell growth factor and related 121-, 145-, 183-, 189-, and 206-amino acid vascular endothelial cell growth factors, as described by Leung et al. Science, 246:1306 (1989), Houck et al. Mol. Endocrin., 5:1806 (1991), and, Robinson & Stringer, Journal of Cell Science, 144(5):853-865 (2001), together with the naturally occurring allelic and processed forms thereof. The term “VEGF” is also used to refer to fragments of the polypeptide, e.g., comprising amino acids 8 to 109 or 1 to 109 of the 165-amino acid human vascular endothelial cell growth factor. Reference to any such forms of VEGF may be identified in the present application, e.g., by “VEGF (8-109),” “VEGF (1-109)” or “VEGF165.” The amino acid positions for a “fragment” native VEGF are numbered as indicated in the native VEGF sequence. For example, amino acid position 17 (methionine) in fragment native VEGF is also position 17 (methionine) in native VEGF. The fragment native VEGF can have binding affinity for the KDR and/or Flt-1 receptors comparable to native VEGF.

An “anti-VEGF antibody” is an antibody that binds to VEGF with sufficient affinity and specificity. The anti-VEGF antibody of the invention can be used as a therapeutic agent in targeting and interfering with diseases or conditions wherein the VEGF activity is involved. An anti-VEGF antibody will usually not bind to other VEGF homologues such as VEGF-B or VEGF-C, nor other growth factors such as PIGF, PDGF or bFGF. See, e.g., U.S. Pat. Nos. 6,582,959 and No. 6,703,020; WO98/45332; WO 96/30046; WO94/10202; EP 0666868B1; U.S. Patent Applications Publication No. 20030206899, No. 20030190317, No. 20030203409, and No. 20050112126; and Popkov et al., Journal of Immunological Methods 288:149-164 (2004). The anti-VEGF antibody “Bevacizumab (BV)”, also known as “rhuMAb VEGF” or “AvastinTM”, is a recombinant humanized anti-VEGF monoclonal antibody generated according to Presta et al. Cancer Res. 57:4593-4599 (1997). It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of Bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies are further described in U.S. Pat. No. 6,884,879.

A “VEGF antagonist” refers to a molecule capable of neutralizing, blocking, inhibiting, abrogating, reducing or interfering with VEGF activities including its binding to one or more VEGF receptors. VEGF antagonists include anti-VEGF antibodies and antigen-binding fragments thereof, receptor molecules and derivatives which bind specifically to VEGF thereby sequestering its binding to one or more receptors, anti-VEGF receptor antibodies and VEGF receptor antagonists such as small molecule inhibitors of the VEGFR tyrosine kinases, and fusions proteins, e.g., VEGF-Trap (Regeneron), VEGF₁₂₁-gelonin (Peregine). VEGF antagonists also include antagonist variants of VEGF, antisense molecules directed to VEGF, RNA aptamers, and ribozymes against VEGF or VEGF receptors.

As used herein, the term “modulate” indicates the ability to control or influence directly or indirectly, and by way of non-limiting examples, can alternatively mean inhibit or stimulate, agonize or antagonize, hinder or promote, and strengthen or weaken. Thus, the term “ANGPTL4 antagonist” refers to an agent that modulates ANGPTL4. Modulators may be organic or inorganic, small to large molecular weight individual compounds, mixtures and combinatorial libraries of inhibitors, agonists, antagonists, and biopolymers such as peptides, nucleic acids, or oligonucleotides. A modulator may be a natural product or a naturally-occurring small molecule organic compound. In particular, a modulator may be a carbohydrate; monosaccharide; oligosaccharide; polysaccharide; amino acid; peptide; oligopeptide; polypeptide; protein; receptor; nucleic acid; nucleoside; nucleotide; oligonucleotide; polynucleotide including DNA and DNA fragments, RNA and RNA fragments and the like; lipid; retinoid; steroid; glycopeptides; glycoprotein; proteoglycan and the like; and synthetic analogues or derivatives thereof, including peptidomimetics, small molecule organic compounds and the like, and mixtures thereof A modulator identified according to the invention is preferably useful in the treatment of a disease disclosed herein.

As used herein, an “antagonist” is a type of modulator and the term refers to an agent that binds a target (e.g., a protein) and can inhibit a one or more functions of the target. For example, an antagonist of a protein can bind the protein and inhibit the binding of a natural or cognate ligand to the protein and/or inhibit signal transduction mediated through the protein.

An “agonist” is a type of modulator and refers to an agent that binds a target and can activate one or more functions of the target. For example, an agonist of a protein can bind the protein and activate the protein in the absence of its natural or cognate ligand.

As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any fragment or derivative of any of the herein described antibodies. In specific embodiments, antibodies may be raised against ANGPTL4 and used as ANGPTL4 antagonists.

The terms “specifically binds to,” “specific for,” and related grammatical variants refer to that binding which occurs between such paired species as antibody/antigen, enzyme/substrate, receptor/agonist, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M.

Optional” or “optionally” means that the subsequently described event or circumstance can or cannot occur, and that the description includes instances where the event or circumstance occurs and instances where it does not.

The terms “subject” and “patient” are used interchangeably here, and are intended to include organisms, e.g., eukaryotes, which are suffering from or afflicted with a disease, disorder or condition associated with ANGPTL4 (e.g., ischemic retinopathies like DME). Examples of subjects or patients include mammals, e.g., humans, dogs, cows, horses, pigs, sheep, goats, cats, mice, rabbits, rats, and transgenic non-human animals. In certain embodiments, the subject or patient is a human, e.g., a human suffering from, at risk of suffering from, or potentially capable of suffering from an ANGPTL4-related disease, disorder or condition.

As used herein, the term “effective,” means adequate to accomplish a desired, expected, or intended result. More particularly, a “therapeutically effective amount” as provided herein refers to an amount of an ANGPTL4 antagonist of the present invention, either alone or in combination with another therapeutic agent, necessary to provide the desired therapeutic effect, e.g., an amount that is effective to prevent, alleviate, or ameliorate symptoms of disease or prolong the survival of the subject being treated. In a specific embodiment, the term “therapeutically effective amount” as provided herein refers to an amount of an ANGPTL4 antagonist, necessary to provide the desired therapeutic effect, e.g., an amount that is effective to prevent, alleviate, or ameliorate symptoms of disease or prolong the survival of the subject being treated. As would be appreciated by one of ordinary skill in the art, the exact amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

The terms “ANGPTL4-related disease, disorder or condition” or “ANGPTL4-mediated disease, disorder or condition,” and the like mean diseases, disorders or conditions associated with ANGPTL4. In certain embodiments, the ANGPTL4-related disease, disorder or condition comprise ischemic retinopathies. In a particular embodiments, the ANGPTL4-related disease, disorder or condition is DME. In other embodiments, the term includes neovascular age-related macular degeneration (AMD), retinopathy of prematurity, incontinentia pigmenti, diabetic retinopathy, retinal vein occlusion, and sickle cell retinopathy, as well as anterior segment neovascular diseases including neovascular glaucoma, corneal neovascularization, pterygia, and pinguecula.

### II. ANGPTL4 Antagonists

In certain embodiments, the ANGPTL4 antagonist is selected from the group consisting of a small molecule, a polypeptide, a nucleic acid molecule, a peptidomimetic, or a combination thereof In a specific embodiment, the agent can be a polypeptide. The polypeptide can, for example, comprise an antibody. In another embodiment, the agent can be a nucleic acid molecule. The nucleic acid molecule can, for example, be an ANGPTL4 inhibitory nucleic acid molecule. The ANGPTL4 inhibitory nucleic acid molecule can comprise a short interfering RNA (siRNA) molecule, a microRNA (miRNA) molecule, or an antisense molecule.

As used herein, an ANGPTL4 inhibitory nucleic acid sequence can be a siRNA sequence or a miRNA sequence. A 21-25 nucleotide siRNA or miRNA sequence can, for example, be produced from an expression vector by transcription of a short-hairpin RNA (shRNA) sequence, a 60-80 nucleotide precursor sequence, which is processed by the cellular RNAi machinery to produce either an siRNA or miRNA sequence. Alternatively, a 21-25 nucleotide siRNA or miRNA sequence can, for example, be synthesized chemically. Chemical synthesis of siRNA or miRNA sequences is commercially available from such corporations as Dharmacon, Inc. (Lafayette, Colo.), Qiagen (Valencia, Calif.), and Ambion, Inc. (Austin, Tex.). An siRNA sequence preferably binds a unique sequence within the ANGPTL4 mRNA with exact complementarity and results in the degradation of the ANGPTL4 mRNA molecule. An siRNA sequence can bind anywhere within the mRNA molecule. An miRNA sequence preferably binds a unique sequence within the ANGPTL4 mRNA with exact or less than exact complementarity and results in the translational repression of the ANGPTL4 mRNA molecule. An miRNA sequence can bind anywhere within the mRNA molecule, but preferably binds within the 3′UTR of the mRNA molecule. Methods of delivering siRNA or miRNA molecules are known in the art. See, e.g., Oh and Park, Adv. Drug Deliv. Rev. 61(10):850-62 (2009); Gondi and Rao, J. Cell. Physiol. 220(2):285-91 (2009); and Whitehead et al., Nat. Rev. Drug Discov. 8(2)129-38 (2009).

As used herein, an ANGPTL4 inhibitory nucleic acid sequence can be an antisense nucleic acid sequence. Antisense nucleic acid sequences can, for example, be transcribed from an expression vector to produce an RNA which is complementary to at least a unique portion of the ANGPTL4 mRNA and/or the endogenous gene which encodes ANGPTL4. Hybridization of an antisense nucleic acid molecule under specific cellular conditions results in inhibition of ANGPTL4 protein expression by inhibiting transcription and/or translation.

The term antibody is used herein in a broad sense and includes both polyclonal and monoclonal antibodies. The term can also refer to a human antibody and/or a humanized antibody. Examples of techniques for human monoclonal antibody production include those described by Cole et al. (Monoclonal Antibodies and Cancer Therapy, Alan R. Liss, p. 77 (1985)) and by Boerner et al. (J. Immunol. 147(1):86-95 (1991)). Human antibodies (and fragments thereof) can also be produced using phage display libraries (Hoogenboom et al., J. Mol. Biol. 227:381 (1991); Marks et al., J. Mol. Biol. 222:581 (1991)). The disclosed human antibodies can also be obtained from transgenic animals. For example, transgenic mutant mice that are capable of producing a full repertoire of human antibodies, in response to immunization, have been described (see, e.g., Jakobovits et al., Proc. Natl. Acad. Sci. USA 90:2551-5 (1993); Jakobovits et al., Nature 362:255-8 (1993); Bruggermann et al., Year in Immunol. 7:33 (1993)).

In other embodiments, an ANGPTL4 antagonist is a small molecule. The term “small molecule organic compounds” refers to organic compounds generally having a molecular weight less than about 5000, 4000, 3000, 2000, 1000, 800, 600, 500, 250 or 100 Daltons, preferably less than about 500 Daltons. A small molecule organic compound may be prepared by synthetic organic techniques, such as by combinatorial chemistry techniques, or it may be a naturally-occurring small molecule organic compound.

Compound libraries may be screened for ANGPTL4 antagonists. A compound library is a mixture or collection of one or more putative modulators generated or obtained in any manner. Any type of molecule that is capable of interacting, binding or has affinity for ANGPTL4 may be present in the compound library. For example, compound libraries screened using this invention may contain naturally-occurring molecules, such as carbohydrates, monosaccharides, oligosaccharides, polysaccharides, amino acids, peptides, oligopeptides, polypeptides, proteins, receptors, nucleic acids, nucleosides, nucleotides, oligonucleotides, polynucleotides, including DNA and DNA fragments, RNA and RNA fragments and the like, lipids, retinoids, steroids, glycopeptides, glycoproteins, proteoglycans and the like; or analogs or derivatives of naturally-occurring molecules, such as peptidomimetics and the like; and non-naturally occurring molecules, such as “small molecule” organic compounds generated, for example, using combinatorial chemistry techniques; and mixtures thereof.

A library typically contains more than one putative modulator or member, i.e., a plurality of members or putative modulators. In certain embodiments, a compound library may comprise less than about 50,000, 25,000, 20,000, 15,000, 10000, 5000, 1000, 500 or 100 putative modulators, in particular from about 5 to about 100, 5 to about 200, 5 to about 300, 5 to about 400, 5 to about 500, 10 to about 100, 10 to about 200, 10 to about 300, 10 to about 400, 10 to about 500, 10 to about 1000, 20 to about 100, 20 to about 200, 20 to about 300, 20 to about 400, 20 to about 500, 20 to about 1000, 50 to about 100, 50 to about 200, 50 to about 300, 50 to about 400, 50 to about 500, 50 to about 1000, 100 to about 200, 100 to about 300, 100 to about 400, 100 to about 500, 100 to about 1000, 200 to about 300, 200 to about 400, 200 to about 500, 200 to about 1000, 300 to about 500, 300 to about 1000, 300 to 2000, 300 to 3000, 300 to 5000, 300 to 6000, 300 to 10,000, 500 to about 1000, 500 to about 2000, 500 to about 3000, 500 to about 5000, 500 to about 6000, or 500 to about 10,000 putative modulators. In particular embodiments, a compound library may comprise less than about 50,000, 25,000, 20,000, 15,000, 10,000, 5,000, 1000, or 500 putative modulators.

A compound library may be prepared or obtained by any means including, but not limited to, combinatorial chemistry techniques, fermentation methods, plant and cellular extraction procedures and the like. A library may be obtained from synthetic or from natural sources such as for example, microbial, plant, marine, viral and animal materials. Methods for making libraries are well-known in the art. See, for example, E. R. Felder, Chimia 1994, 48, 512-541; Gallop et al., J. Med. Chem. 1994, 37, 1233-1251; R. A. Houghten, Trends Genet. 1993, 9, 235-239; Houghten et al., Nature 1991, 354, 84-86; Lam et al., Nature 1991, 354, 82-84; Carell et al., Chem. Biol. 1995, 3, 171-183; Madden et al., Perspectives in Drug Discovery and Design 2, 269-282; Cwirla et al., Biochemistry 1990, 87, 6378-6382; Brenner et al., Proc. Natl. Acad. Sci. USA 1992, 89, 5381-5383; Gordon et al., J. Med. Chem. 1994, 37, 1385-1401; Lebl et al., Biopolymers 1995, 37 177-198; and references cited therein. Compound libraries may also be obtained from commercial sources including, for example, from Maybridge, ChemNavigator.com, Timtec Corporation, ChemBridge Corporation, A-Syntese-Biotech ApS, Akos-SC, G & J Research Chemicals Ltd., Life Chemicals, Interchim S.A., and Spectrum Info. Ltd.

### III. Methods of Using ANGPTL4 Antagonists

The ANGPTL4 antagonists described herein have in vitro and in vivo diagnostic and therapeutic utilities. For example, these molecules can be administered to cells in culture, e.g., in vitro or in vivo, or in a subject, e.g., in vivo, to treat, prevent or diagnose a variety of ANGPTL4-mediated diseases, disorders or conditions. ANGPTL4 antagonists are particularly suitable for treating human patients suffering from “ANGPTL4-related disorders,” meaning those diseases and conditions associated with aberrant ANGPTL4. Aberrant upregulation of ANGPTL4 would be particularly amendable to treatment by the administration of antagonizing ANGPTL4 modulators. Conversely, aberrant downregulation of ANGPTL4 would be particularly amendable to treatment by the administration of agonizing ANGPTL4 modulators. In certain embodiments, the ANGPTL4 antagonizing modulators of the present invention are capable of reducing or inhibiting vascular permeability in ischemic retinopathies including, for example, DME. In other embodiments, the ischemic retinopathies include, but are not limited to, neovascular age-related macular degeneration (AMD), retinopathy of prematurity, incontinentia pigmenti, diabetic retinopathy, retinal vein occlusion, and sickle cell retinopathy.

Also within the scope of the invention are kits comprising the compositions of the invention and instructions for use. In one embodiment, the kit comprises an anti-ANGPTL4 antibody. The kit can further contain a least one additional reagent, or one or more additional antibodies (e.g., an antibody having a complementary activity which binds to an epitope on the target antigen distinct from the first antibody). Kits typically include a label indicating the intended use of the contents of the kit. The term label includes any writing, or recorded material supplied on or with the kit, or which otherwise accompanies the kit.

### IV. Pharmaceutical Compositions and Administration

Accordingly, a pharmaceutical composition of the present invention may comprise an effective amount of an ANGPTL4 antagonist. As used herein, the term “effective,” means adequate to accomplish a desired, expected, or intended result. More particularly, an “effective amount” or a “therapeutically effective amount” is used interchangeably and refers to an amount of an ANGPTL4 antagonist, perhaps in further combination with yet another therapeutic agent, necessary to provide the desired “treatment” (defined herein) or therapeutic effect, e.g., an amount that is effective to prevent, alleviate, treat or ameliorate symptoms of a disease or prolong the survival of the subject being treated. In particular embodiments, the pharmaceutical compositions of the present invention are administered in a therapeutically effective amount to treat patients suffering from an ANGPTL4-mediated disease, disorder or condition. In a more specific embodiment, the pharmaceutical compositions of the present invention are administered in an effective amount to treat patients suffering from ischemic retinopathies. In a specific embodiment, the ischemic retinopathy is diabetic macular edema (DME). As would be appreciated by one of ordinary skill in the art, the exact low dose amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

The pharmaceutical compositions of the present invention are in biologically compatible form suitable for administration in vivo for subjects. The pharmaceutical compositions can further comprise a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which an ANGPTL4 antagonist is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water may be a carrier when the pharmaceutical composition is administered orally. Saline and aqueous dextrose may be carriers when the pharmaceutical composition is administered intravenously. Saline solutions and aqueous dextrose and glycerol solutions may be employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The pharmaceutical composition may also contain minor amounts of wetting or emulsifying agents, or pH buffering agents.

The pharmaceutical compositions of the present invention can take the form of solutions, suspensions, emulsions, tablets, pills, capsules, powders, sustained-release formulations and the like. The composition can be formulated as a suppository, with traditional binders and carriers such as triglycerides. Oral formulation may include standard carriers such as pharmaceutical grades of mannitol, lactose, starch, magnesium stearate, sodium saccharine, cellulose, magnesium carbonate, etc. In a specific embodiment, a pharmaceutical composition comprises an effective amount of an ANGPTL4 antagonist together with a suitable amount of a pharmaceutically acceptable carrier so as to provide the form for proper administration to the patient. The formulation should suit the mode of administration.

The pharmaceutical compositions of the present invention may be administered by any particular route of administration including, but not limited to intraocular oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intraosseous, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means. Most suitable routes are oral administration or injection. In certain embodiments, an intraocular injection is preferred.

In general, the pharmaceutical compositions comprising an ANGPTL4 antagonist may be used alone or in concert with other therapeutic agents at appropriate dosages defined by routine testing in order to obtain optimal efficacy while minimizing any potential toxicity. The dosage regimen utilizing a pharmaceutical composition of the present invention may be selected in accordance with a variety of factors including type, species, age, weight, sex, medical condition of the patient; the severity of the condition to be treated; the route of administration; the renal and hepatic function of the patient; and the particular pharmaceutical composition employed. A physician of ordinary skill can readily determine and prescribe the effective amount of the pharmaceutical composition (and potentially other agents including therapeutic agents) required to prevent, counter, or arrest the progress of the condition.

Optimal precision in achieving concentrations of the therapeutic regimen (e.g., pharmaceutical compositions comprising an ANGPTL4 antagonist, optionally in combination with another therapeutic agent) within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. The dosages of a pharmaceutical composition disclosed herein may be adjusted when combined to achieve desired effects. On the other hand, dosages of the pharmaceutical compositions and various therapeutic agents may be independently optimized and combined to achieve a synergistic result wherein the pathology is reduced more than it would be if either was used alone.

In particular, toxicity and therapeutic efficacy of a pharmaceutical composition disclosed herein may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effect is the therapeutic index and it may be expressed as the ratio LD₅₀/ED₅₀. Pharmaceutical compositions exhibiting large therapeutic indices are preferred except when cytotoxicity of the composition is the activity or therapeutic outcome that is desired. Although pharmaceutical compositions that exhibit toxic side effects may be used, a delivery system can target such compositions to the site of affected tissue in order to minimize potential damage to uninfected cells and, thereby, reduce side effects. Generally, the pharmaceutical compositions of the present invention may be administered in a manner that maximizes efficacy and minimizes toxicity.

Data obtained from cell culture assays and animal studies may be used in formulating a range of dosages for use in humans. The dosages of such compositions lie preferably within a range of circulating concentrations that include the ED₅₀ with little or no toxicity. The dosage may vary within this range depending upon the dosage form employed and the route of administration utilized. For any composition used in the methods of the invention, the therapeutically effective dose may be estimated initially from cell culture assays. A dose may be formulated in animal models to achieve a circulating plasma concentration range that includes the IC₅₀ (the concentration of the test composition that achieves a half-maximal inhibition of symptoms) as determined in cell culture. Such information may be used to accurately determine useful doses in humans. Levels in plasma may be measured, for example, by high performance liquid chromatography.

Moreover, the dosage administration of the compositions of the present invention may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See WO 00/67776, which is entirely expressly incorporated herein by reference.

More specifically, the pharmaceutical compositions may be administered in a single daily dose, or the total daily dosage may be administered in divided doses of two, three, or four times daily. In the case of oral administration, the daily dosage of the compositions may be varied over a wide range from about 0.1 ng to about 1,000 mg per patient, per day. The range may more particularly be from about 0.001 ng/kg to 10 mg/kg of body weight per day, about 0.1-100 μg, about 1.0-50 μg or about 1.0-20 mg per day for adults (at about 60 kg).

The daily dosage of the pharmaceutical compositions may be varied over a wide range from about 0.1 ng to about 1000 mg per adult human per day. For oral administration, the compositions may be provided in the form of tablets containing from about 0.1 ng to about 1000 mg of the composition or 0.1, 0.2, 0.5, 1.0, 2.0, 5.0, 10.0, 15.0, 100, 150, 200, 250, 300, 350, 400, 450, 500, 550, 600, 650, 700, 800, 900, or 1000 milligrams of the composition for the symptomatic adjustment of the dosage to the patient to be treated. An effective amount of the pharmaceutical composition is ordinarily supplied at a dosage level of from about 0.1 ng/kg to about 20 mg/kg of body weight per day. In one embodiment, the range is from about 0.2 ng/kg to about 10 mg/kg of body weight per day. In another embodiment, the range is from about 0.5 ng/kg to about 10 mg/kg of body weight per day. The pharmaceutical compositions may be administered on a regimen of about 1 to about 10 times per day.

In the case of injections, it is usually convenient to give in an amount of about 0.0001 μg-30 mg, about 0.01 μg-20 mg or about 0.01-10 mg per day to adults (at about 60 kg). In the case of other animals, the dose calculated for 60 kg may be administered as well.

Doses of a pharmaceutical composition of the present invention can optionally include 0.0001 μg to 1,000 mg/kg/administration, or 0.001 μg to 100.0 mg/kg/administration, from 0.01 μg to 10 mg/kg/administration, from 0.1 μg to 10 mg/kg/administration, including, but not limited to, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53,54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration or any range, value or fraction thereof.

As a non-limiting example, treatment of subjects can be provided as a one-time or periodic dosage of a composition of the present invention 0.1 ng to 100 mg/kg such as 0.0001, 0.001, 0.01, 0.1 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Specifically, the pharmaceutical compositions of the present invention may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

More specifically, the pharmaceutical compositions may be administered at least once a day for about 2 days, at least once a day for about 3 days, at least once a day for about 4 days, at least once a day for about 5 days, at least once a day for about 6 days, at least once a day for about 7 days, at least once a day for about 8 days, at least once a day for about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

Alternatively, the pharmaceutical compositions may be administered about once every day, about once every 2 days, about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every 18 days, about once every 19 days, about once every 20 days, about once every 21 days, about once every 22 days, about once every 23 days, about once every 24 days, about once every 25 days, about once every 26 days, about once every 27 days, about once every 28 days, about once every 29 days, about once every 30 days, or about once every 31 days.

The pharmaceutical compositions of the present invention may alternatively be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks.

Alternatively, the pharmaceutical compositions of the present invention may be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months.

Alternatively, the pharmaceutical compositions may be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a week for about 13 weeks, at least once a week for about 14 weeks, at least once a week for about 15 weeks, at least once a week for about 16 weeks, at least once a week for about 17 weeks, at least once a week for about 18 weeks, at least once a week for about 19 weeks, or at least once a week for about 20 weeks.

Alternatively the pharmaceutical compositions may be administered at least once a week for about 1 month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months.

The pharmaceutical compositions may further be combined with one or more additional therapeutic agents. The second therapeutic agent can be another agent useful for treating ischemic retinopathies. For example, the second therapeutic agents can be a VEGF antagonist. In other embodiments, the second therapeutic agents can be a HIF-1α antagonist. A combination therapy regimen may be additive, or it may produce synergistic results (e.g., a reduction of ischemic retinopathy (e.g., DME) greater than expected for the combined use of the two agents).

The compositions can be administered simultaneously or sequentially by the same or different routes of administration. The determination of the identity and amount of the pharmaceutical compositions for use in the methods of the present invention can be readily made by ordinarily skilled medical practitioners using standard techniques known in the art. In specific embodiments, an ANGPTL4 antagonist of the present invention can be administered in combination with an effective amount of another therapeutic agent (e.g., VEGF antagonist).

In various embodiments, the ANGPTL4 antagonist of the present invention in combination with an another therapeutic agent (e.g., a VEGF antagonist) may be administered at about the same time, less than 1 minute apart, less than 2 minutes apart, less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In particular embodiments, two or more therapies are administered within the same patent visit.

In certain embodiments, the ANGPTL4 antagonist of the present invention in combination with another therapeutic agent (e.g., a VEGF antagonist) is cyclically administered. Cycling therapy involves the administration of a first therapy (e.g., the ANGPTL4 antagonist) for a period of time, followed by the administration of a second therapy (e.g., another therapeutic agent) for a period of time, optionally, followed by the administration of perhaps a third therapy for a period of time and so forth, and repeating this sequential administration, e.g., the cycle, in order to reduce the development of resistance to one of the therapies, to avoid or reduce the side effects of one of the therapies, and/or to improve the efficacy of the therapies. In certain embodiments, the administration of the combination therapy of the present invention may be repeated and the administrations may be separated by at least 1 day, 2 days, 3 days, 5 days, 10 days, 15 days, 30 days, 45 days, 2 months, 75 days, 3 months, or at least 6 months.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Cell Culture and Reagents. MIO-M1 cells were a generous gift from Astrid Limb (University College London Institute of Ophthalmology). Isolation of primary Müller cells was performed as previously described (51). Primary human dermal microvascular endothelial cells (HMVEC) were obtained from Lonza and cultured according the manufacter's protocols. Recombinant ANGPTL4 and VEGF, and ANGPTL4 and VEGF ELISA kits were obtained from R&D Systems. The KDR inhibitor, SU1498, and rapamycin were obtained from Calbiochem. Digoxin was obtained from Sigma. Predesigned control (Scrambled) and ANGPTL4 and HIF-1β siRNA sequences were obtained from Qiagen. AdLacZ and AdCAS have been previously described (26). Hypoxia chambers were used to expose MIO-M1 cells (1% oxygen) and primary murine Müller cells (3% oxygen; exposure of primary murine Müller cells to lower oxygen concentrations resulted in cell death).

Mice. Eight-week old pathogen-free female C57BL/6 mice (The Jackson Laboratory), female athymic nu-n mice (Harlan Sprague Dawley) and timed pregnant C57BL/6 mice (E14) (Charles River Laboratories) were treated in accordance with the Association for Research in Vision and Ophthalmology Statement for the Use of Animals in Ophthalmic and Vision Research and the guidelines of the Johns Hopkins University Animal Care and Use Committee.

Oxygen-Induced Retinopathy (OIR). Oxygen-Induced Retinopathy experiments were performed as previously described (13). In brief, C57BL/6 mice were placed in 75% O₂ at postnatal day P7. At P12 the mice were returned to room air. A subset of mice was given daily intraperitoneal injection of vehicle or 2 mg/kg of digoxin. Retinas were collected from mice at P12, P13, P14, and/or P17.

Intraocular Infections. Intraocular injections with 1 μl of rmVEGF or rmANGPTL4 (200 ng/ul) were performed with a nanofil syringe (World Precision Instruments) using a 36G beveled needle. Intraocular injections with 1 μl of AdLacZ or AdCAS (2×10¹² VP/mL) were performed with pulled-glass micropipettes and a Harvard pump microinjection apparatus as previously described (52).

Western Blot and ELISA. Cell and neurosensory retina lysates were subjected to 4%-15% gradient SDS-PAGE (Invitrogen) Immunoblot assays were performed with primary antibodies specifically recognizing HIF-1α (Abcam, Ab2185; MW 120 kDa for endogenous and 100 kDa for CA5 mutant), ANGPTL4 (Millipore) and GAPDH (Fitzgerald). Levels of secreted VEGF and ANGPTL4 were measured in media conditioned by MIO-M1 cells using DuoSet human VEGF or ANGPTL4 ELISA kits (R&D System).

Quantitative Real-Time RT-PCR. RNA was isolated from culture cells or retinas with RNeasy Mini Kit (Qiagen) and cDNA prepared using MuLV Reverse Transcriptase (Applied Biosystems, Carlsbad, Calif.). Quantitative real-time PCR was performed with Power SYBR Green PCR Master Mix (Applied Biosystems) and MyiQ Real-Time PCR Detection System (Bio Rad). Normalization was done using cyclophilin A for mouse tissue and cell lines and β-actin for human cell lines. Primers for qPCR include: human VEGF, forward—GGGCAGAATCATCACGAAGT (SEQ ID NO:1) and reverse—TGGTGATGTTGGACTCCTCA (SEQ ID NO:2); human ANGPTL4, forward—GGACACGGCCTATAGCCTG (SEQ ID NO:3) and reverse—CTCTTGGCGCAGTTCTTGTC (SEQ ID NO:4); human β-actin, forward—CTCTTCCAGCCTTCCTTCCT (SEQ ID NO:5) and reverse—AGCACTGTGTTGGCGTACAG(SEQ ID NO:6) ; mouse VEGF, forward—TTACTGCTGTACCTCCACC (SEQ ID NO:7) and reverse—ACAGGACGGCTTGAAGATG(SEQ ID NO:8); mouse ANGPTL4, forward—TTGGTACCTGTAGCCATTCC (SEQ ID NO:9) and reverse—GAGGCTAAGAGGCTGCTGTA (SEQ ID NO:10); mouse cyclophilin A, forward—AGCATACAGGTCCTGGCATC (SEQ ID NO:11) and reverse—TTCACCTTCCCAAAGACCAC (SEQ ID NO:12).

Permeability Assays. In vitro permeability assay was performed as previously described (48). Briefly, HMVEC were seeded on collagen-coated transwells (3-mm-size pore, PTFE; Corning), and allowed to grow as a 3-d-old mature monolayer. Following overnight starvation, 500 μL and 100 μL of conditioned medium were added for 30 min (37° C.) to the bottom and top chamber, respectively. A total of 100 μL of 1 mg/mL FITC dextran (molecular weight=40,000; Invitrogen) was added for 30 min. Fluorescence was quantified using a SpectraMax M5 Microplate Reader (Molecular Devices) with excitation at 494 nm and emission at 521 nm.

In vivo permeability was assessed using a modified Miles assay as previously described (48). Briefly, Evan Blue dye was injected into mouse tail veins (200 μL of 12 mg/ml solution in PBS). After 5 minutes, animals were anesthetized using Tribromoethanol. 20 ul 0.2 ug VEGF or 0.2 ug ANGPTL4 was injected intradermally into the right ear (PBS was injected into left ear as a control). After 8 minutes, photographs were taken. Levels of Evans blue dye was then extracted from mouse ear in 1 mL of formamide at 55° C. for 16 hours and dye content quantified at 610 nm using a spectrophotometer (48).

Quantitation of vascular permeability in the retina was assessed by examining 4 high powered fields of the posterior retina from three animals and counting the number of CD31-labled inner retina vessels in the posterior retina that had visible adjacent extravascular albumin (detected by immunofluorescence) as a percent of the total vessels in each field.

MicroArray. Briefly, MIOM1 cells were treated with or without hypoxia for 8 hours and the mRNA was extracted with RNeasy Mini Kit (Qiagen). 300 to 500 ng of the mRNA was used for microarray assay. The MicroArray assay was performed using the Affymetix Human Gene 1.0ST MicroArray by the Johns Hopkins Deep Sequencing & Microarray Core Facility. The fold of increase of an individual gene expression under hypoxia treatment was calculated using the formula 2⁽ⁿ²⁻ⁿ¹⁾, where n2 is the reading of the hypoxia sample and n1 is the reading of the normoxia sample.

Immunohistochemistry and Immunofluorescence. Immunohistochemical detection of extravascular albumin (Cedarlane-Nordic) was performed on cryopreserved mouse tissue sections using a nitroblue tetrazolium (NBT) development system using Streptavidin alkaline phosphatase (APase) as previously described (53) Immunohistochemical detection of HIF-1 alpha (Abcam), human and murine ANGPTL4 (Abcam and Lifespan BioSciences, respectively), VEGF (Santa Cuz), CD34 (Covance), and GFAP (Sigma) were performed in paraffin embedded human tissue (obtained from the Wilmer Eye Institute Ocular Pathology Archives with approval from the Johns Hopkins School of Medicine Internal Review Board) and mice cryopreserved tissue using ABC system (Dako) as previously described (4).

Immunofluorescence detection of CD31 (BD Pharmingen), Hypoxy-probe (HPI), GFAP, (Sigma), HIF-1 alpha (Abcam), vimentin (Abcam), and albumin (Cedarlane-Nordic) was performed on retina flat mounts or cryopreserved mouse tissue sections as previously described (55-57) Immunodetection was performed using goat anti-mouse Alexa F 555, goat anti-rabbit Alexa F 488, and goat anti-rat Alexa F 647 (Invitrogen) associated with DAPI (Invitrogen). Images were captured using the Zeiss confocal microscope meta 710 LSM (Carl Zeiss Inc., Thornwood, N.Y.).

Statistical Analysis. In all cases, results are shown as a mean value±SD from at least three independent experiments. Western blot scans are representative of at least three independent experiments. Statistical analysis was performed with Prism 4.2 software (GraphPad). ANOVA test: ***, p<0.001; **, p<0.01; *, p<0.05.

### Results

### EXAMPLE 1

### HIF-1α Accumulation and Müller Glial Cell Injury/Activation Co-localize in the Ischemic Inner Retina in the OIR Model

Microvascular complications in diabetic patients are caused by prolonged exposure to high glucose levels. Mouse models in which the hyperglycemic state is replicated have proven essential for studying the early stages of diabetic eye disease, to be sure. However, these models do not adequately reproduce the retinal nonperfusion that results in the release of growth factors that, in turn, promote the vascular permeability characteristic of patients with DME (1). Although no animal model has yet been found to demonstrate all of the microvascular complications associated with patients with diabetic eye disease, the oxygen induced retinopathy (OIR) mouse model faithfully reproduces the inner retina ischemia (nonperfusion) observed in these patients and has proven to be an important tool for studying the pathogenesis of ischemic retinopathies (13).

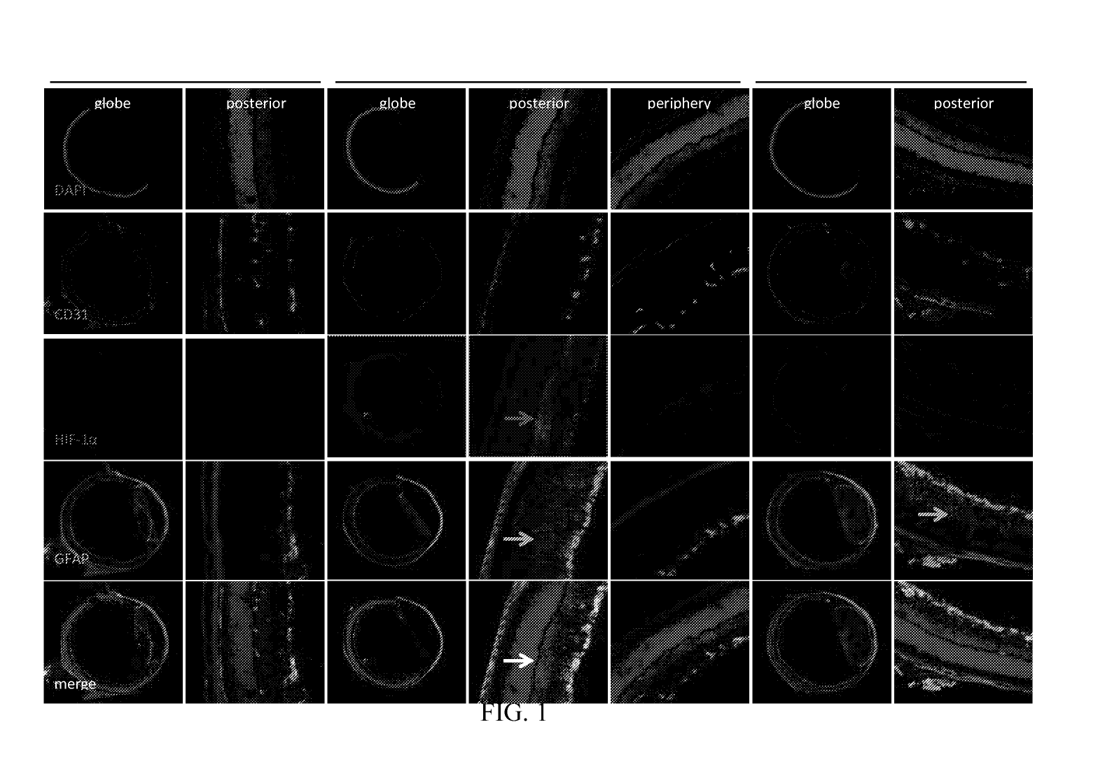

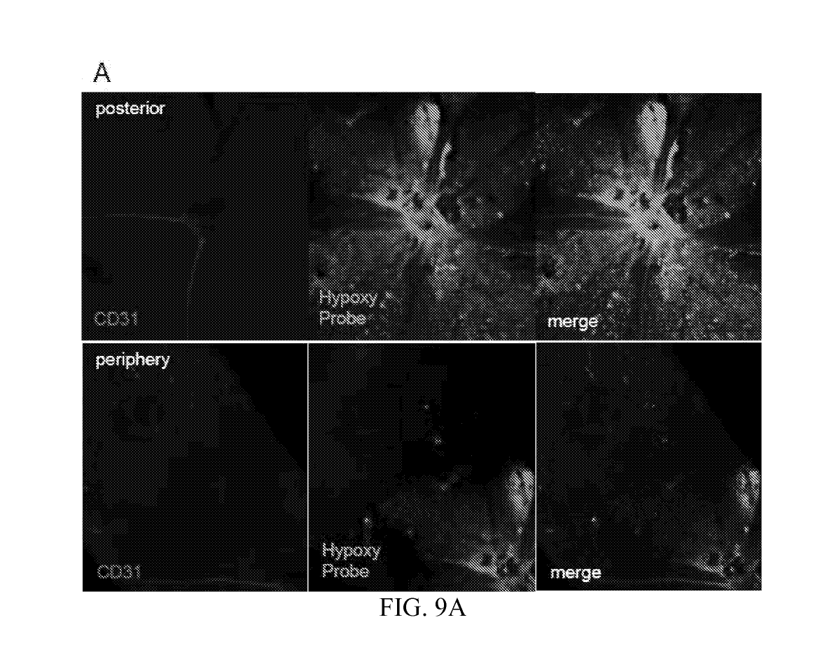

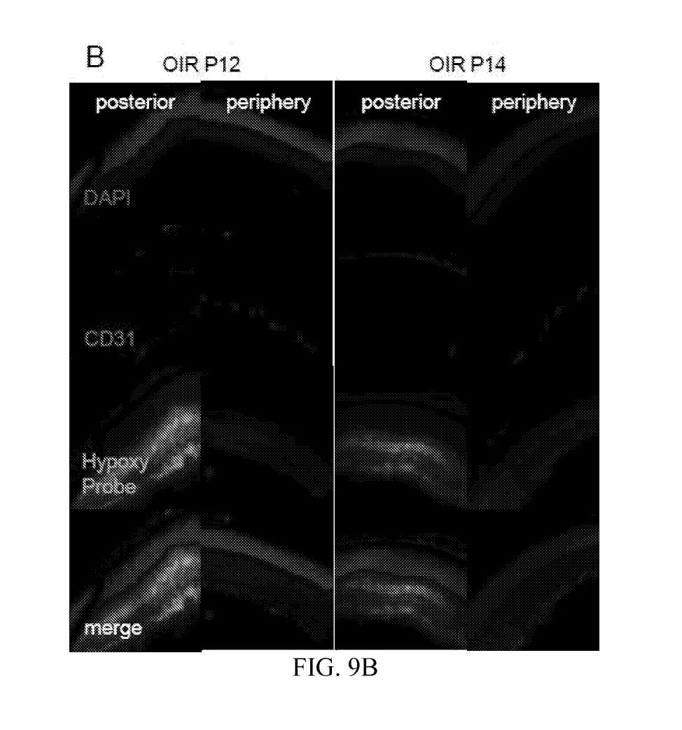

The inner retina is composed of several cell types, including neurons (retinal ganglion cells and bipolar cells, horizontal cells and amacrine cells) and glial cells (astrocytes and Müller cells). However, the cell(s) responsible for elaborating HIF-1-target proteins that contribute to ischemic retinal disease remain unclear. To better understand the molecular pathogenesis of DME, we first set out to examine HIF-1α protein stabilization in retinal cells using the oxygen-induced retinopathy (OIR) mouse model (13), which has proven to be an important tool for studying the pathogenesis of ischemic retinopathies. In OIR mice, vaso-obliteration of the posterior retinal vasculature during the hyperoxia phase (P7-P 12) results in retinal hypoxia in the posterior—but not the peripheral—retina (FIGS. 9 and (14)). HIF-1α protein levels were increased within 24 hours of returning mice to normoxic conditions in the posterior (hypoxic) inner retina within the inner nuclear layer (FIG. 1), as has been previously described (15).

Increased HIF-1α protein levels in the inner retina localized to an area with an increase in the expression of the intermediate filament protein, glial fibrillary acidic protein (GFAP) (FIG. 10A). GFAP is expressed in astrocytes, but also in injured or “activated” retinal Müller glial cells in response to injury during different pathological conditions (including ischemia, trauma, retinal degeneration, and glaucoma). Of interest, in ischemic diseases affecting the brain (e.g., stroke), astrocytes—glial cells previously thought to contribute only a supportive or structural role—have recently emerged as central players in the response to ischemia (16). In this context, glial cells play an essential role in the angiogenic response, producing key secreted factors that act in concert to help acclimate the neurons (and brain) to conditions of reduced oxygen tension. To further explore whether Müller glial cells may play an analogous role in ischemic retinopathies, we examined expression of GFAP in the posterior (hypoxic) inner retina compared to the intermediate and peripheral inner retina in the OIR model. We observed a marked increase in expression of GFAP within hypoxic Müller cells in the inner retina in the ischemic posterior-intermediate but not in the perfused peripheral retina (FIG. 10). Interestingly, GFAP expression was not affected by administration of digoxin, an inhibitor of HIF-1α translation (17), suggesting that GFAP expression is a result of Müller cell injury from hypoxia, but is independent of HIF-1α transcriptional activity (FIGS. 1 and 10).

### EXAMPLE 2

### Hypoxia Upregulates HIF and VEGF in Injured Müller Glial Cells

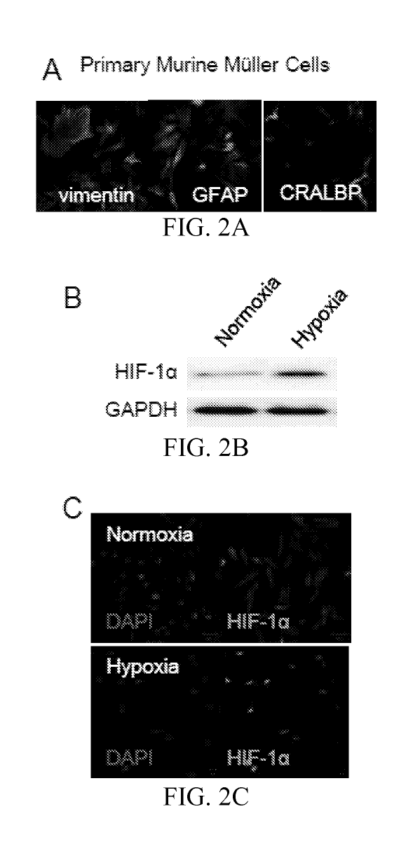

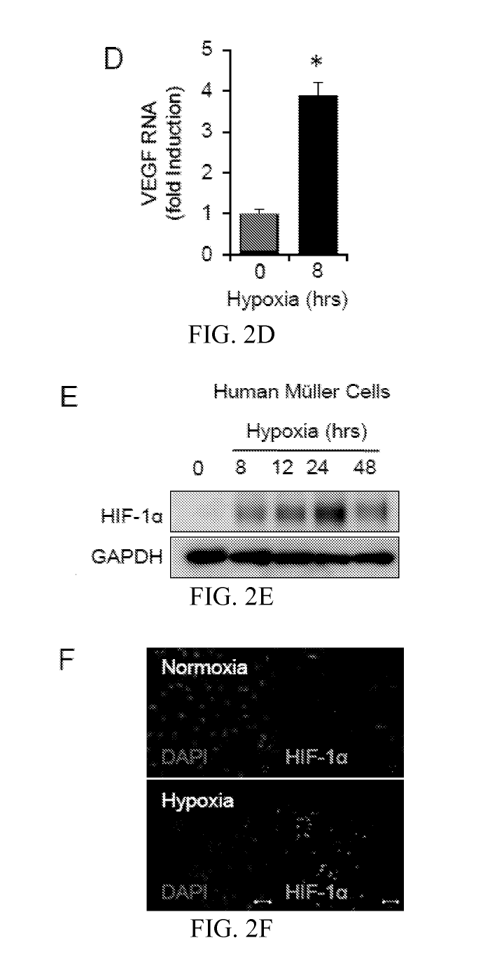

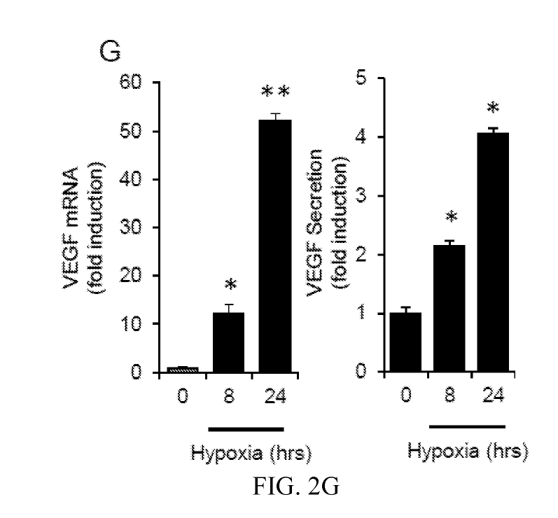

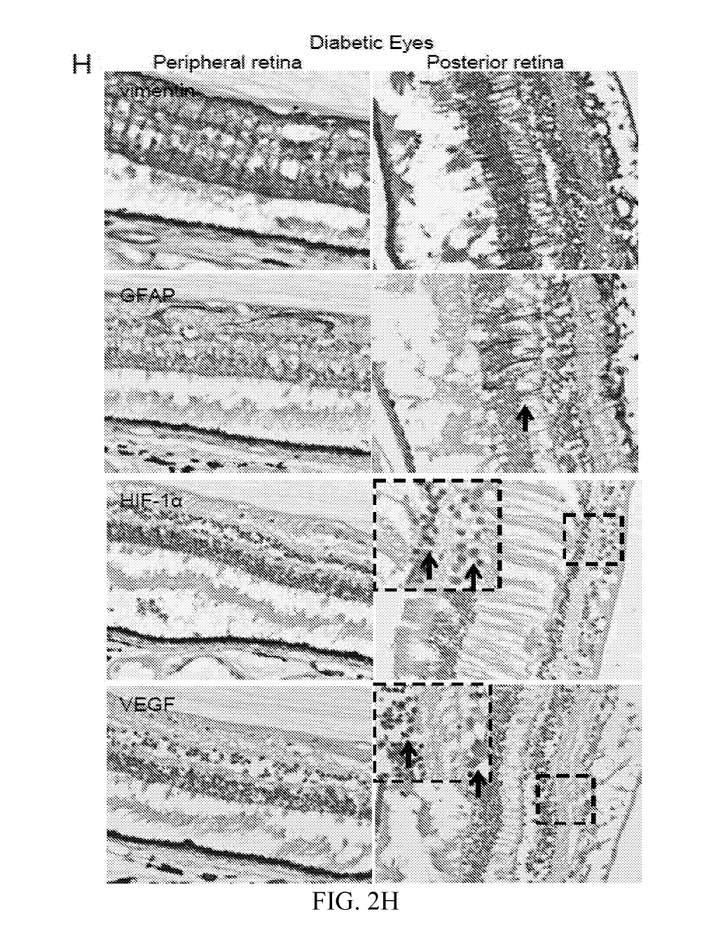

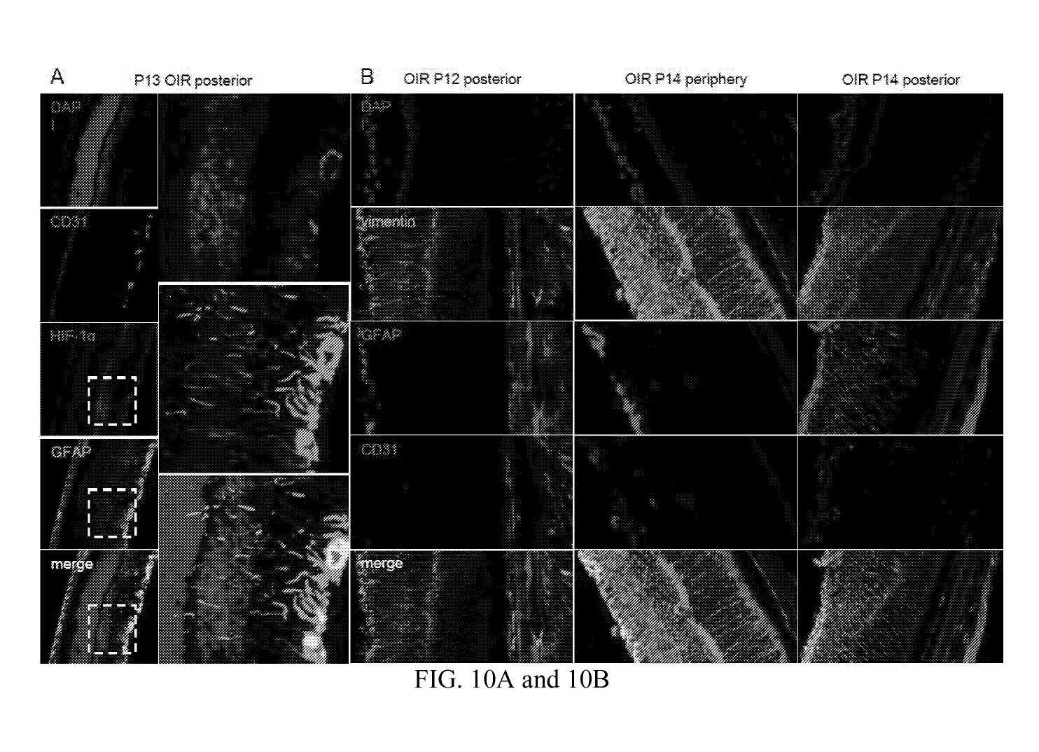

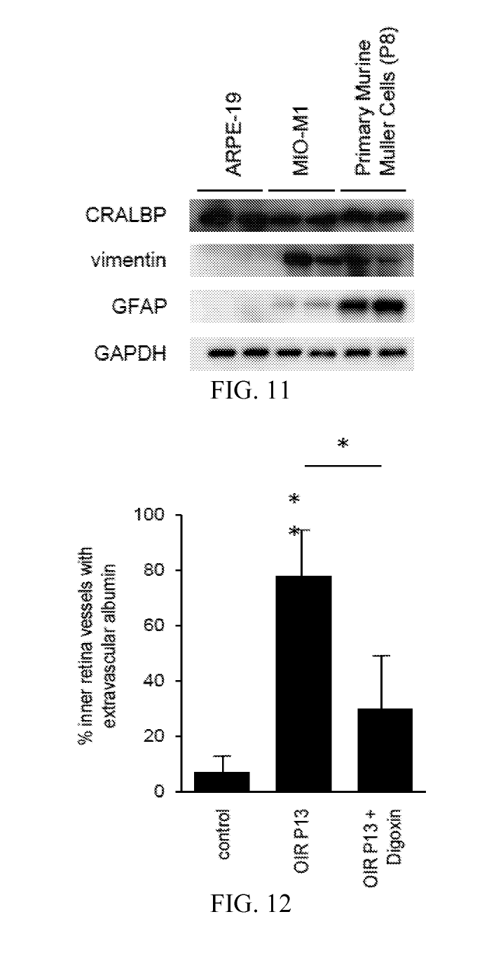

To directly assess the response of retinal Müller cells to hypoxia, we isolated primary Müller cell cultures (>95% pure) from the neurosensory retinas of P0-P5 C57BL/6 mice (FIG. 2A). These cells maintained a Müller cell phenotype for over 8 passages, as demonstrated by the expression of key Müller cell markers, including vimentin, CRALBP and GFAP (FIG. 11). Primary murine Müller cells responded to hypoxia (3% O₂) with an increase in HIF-1α protein stability and nuclear localization (FIGS. 2B and C), and an increase in the mRNA levels of the HIF-1 target gene, Vegf (FIG. 2D). To confirm a role for Müller cells in the hypoxic response in humans, we took advantage of the availability of a previously characterized immortalized human Müller (MIO-M1) cell line (18). Similar to primary murine Müller cells, exposure of MIO-M1 cells to hypoxia resulted in an increase in HIF-1α protein stability and nuclear localization (FIGS. 2E and F), resulting in an increase in VEGF mRNA levels and secreted protein (FIG. 2G).

We then examined retinal tissue from patients with known DME to determine whether ischemic (injured) Müller cells upregulate HIF-1 and its target genes in these patients. Similar to the OIR model, injured (GFAP-expressing) Müller cells were detected in the ischemic (posterior) retina, but not in the perfused peripheral retina (FIG. 2H). HIF-1α and VEGF also localized to the posterior inner retina, but were not detected in the peripheral retina (FIG. 2H).

### EXAMPLE 3

### Inhibition of HIF Blocks Edema in Ischemic Retinal Disease In Vivo

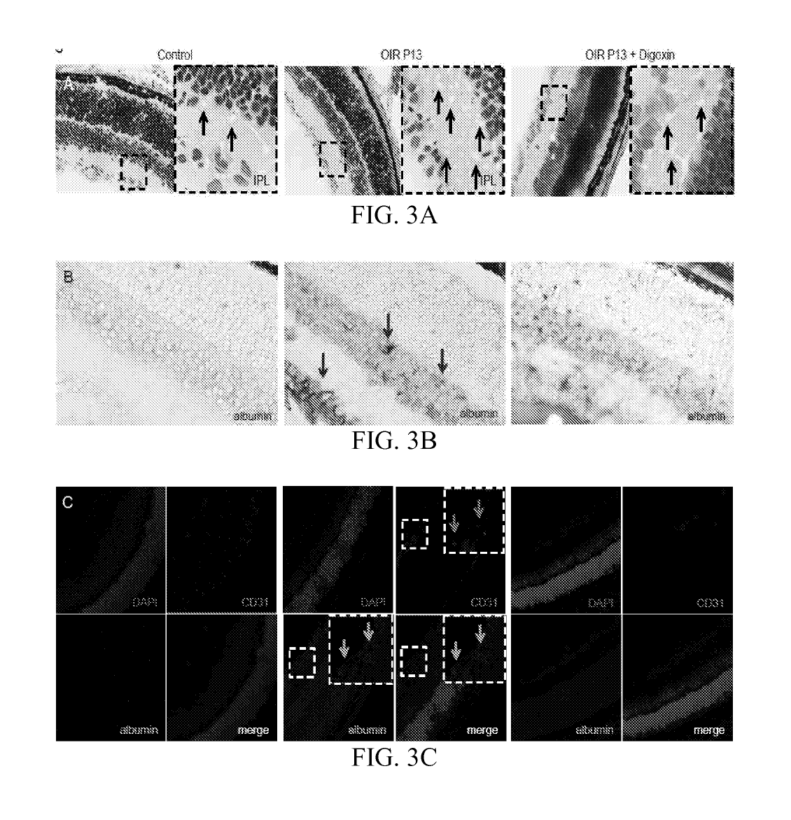

We next set out to determine whether inhibition of HIF-1α could reduce edema in ischemic retinopathies in vivo. Although the OIR model has been used extensively as a model for retinal neovascularization (14), we observed that this model results in increased vascular permeability, with leakage of plasma (FIG. 3A) and the plasma protein, albumin (FIG. 3B and C) into the interstitial tissue. The administration of digoxin to inhibit HIF-1α translation resulted in a decrease in vascular permeability (FIG. 3A-C and 12), demonstrating that HIF-1 is required for the promotion of vascular permeability, and supporting a therapeutic role for the inhibition of HIF-1α for the treatment of DME.

### EXAMPLE 4

### VEGF Alone is Not Sufficient to Explain the Induction of Vascular Permeability Mediated by HIF-1 in Hypoxic Müller Cells

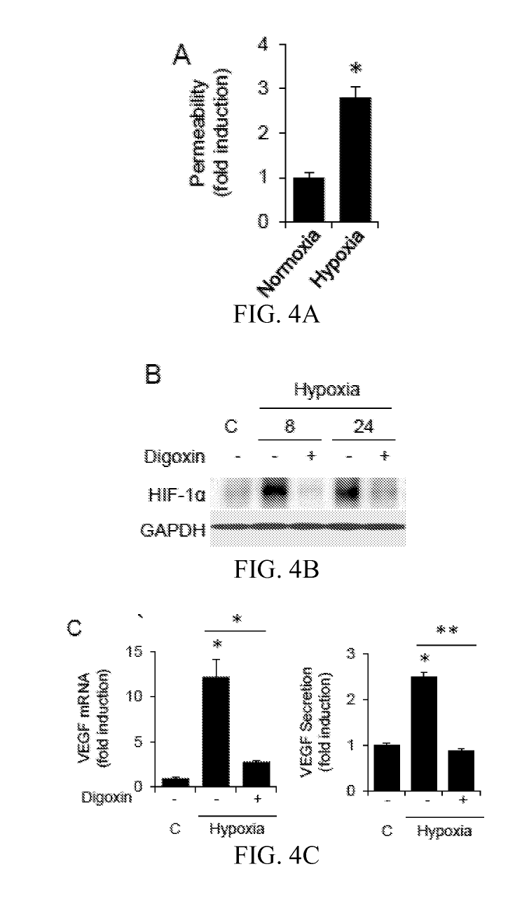

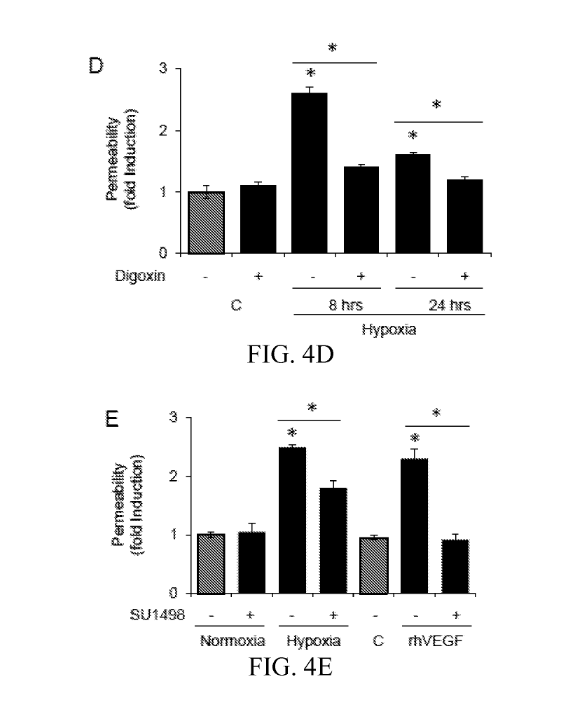

To further examine the contribution of secreted factors elaborated by hypoxia-treated Müller cells to vessel leakage, we next treated monolayers of human dermal microvascular endothelial cells (HMVECs) with conditioned medium from MIO-M1 cells exposed to hypoxia and assessed the promotion of endothelial cell permeability as determined by passage of FITC-dextran. Conditioned medium from the MIO-M1 cells exposed to hypoxia increased endothelial cell permeability by almost three-fold compared to MIO-M1 cells cultured under non-hypoxic conditions (FIG. 4A).

HIF-1 plays a major role in regulating the ubiquitous transcriptional response to hypoxia. Nonetheless, a number of other transcription factors (e.g., NFκB, CREB, AP-1, p53, SP-1, SP-3) are also activated either directly or indirectly by hypoxia. We therefore set out to confirm that HIF-1-dependent gene expression in hypoxic MIO-M1 cells was primarily responsible for the promotion of endothelial cell permeability. Pre-treatment of MIO-M1 cells with digoxin blocked hypoxic induction of HIF-1α protein accumulation (FIG. 4B). This, in turn, inhibited VEGF mRNA expression and protein secretion (FIG. 4C), and potently blocked the property of the conditioned media to promote an increase in endothelial cell permeability (FIG. 4D).

To assess whether the effect of digoxin on the promotion of endothelial cell leakage by hypoxic Müller cells was mediated mainly by its inhibition of HIF-1-upregulation of VEGF, we pre-treated endothelial cells with the VEGF receptor-2 (VEGFR2 or KDR) inhibitor, SU1498. While effective doses of SU1498 completely blocked the induction of endothelial cell permeability promoted by 100 ng of recombinant human (rh)VEGF—two orders of magnitude higher than the levels secreted by hypoxic MIO-M1 cells—it resulted in only a partial inhibition of endothelial cell permeability by conditioned media from hypoxia-treated MIO-M1 cells (FIG. 4E). These results suggest that inhibition of HIF-1α may be a more potent therapeutic approach for the treatment of macular edema than inhibition of VEGF alone. Our findings further suggest that, in addition to VEGF, other HIF-dependent secreted factors may participate in the promotion of vascular permeability by hypoxic Müller cells.

### EXAMPLE 5

### ANGPTL4 is Unregulated by HIF-1 in Hypoxic Cultures of Müller Cells

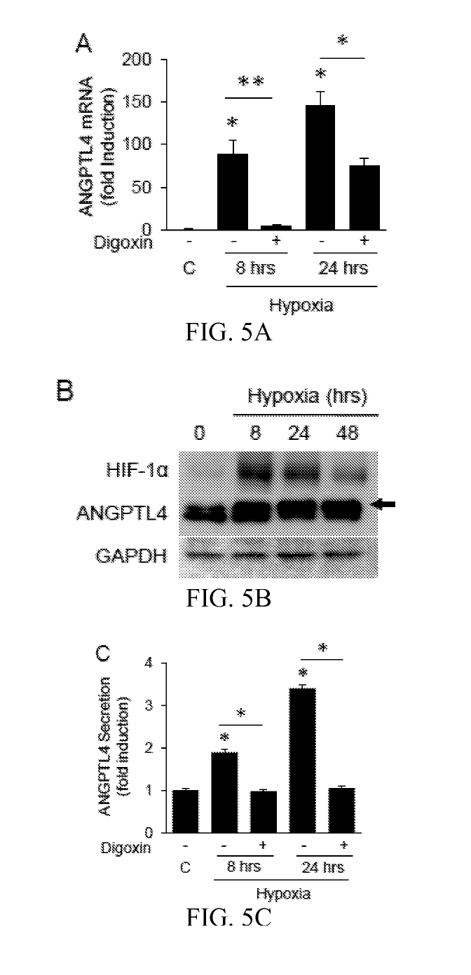

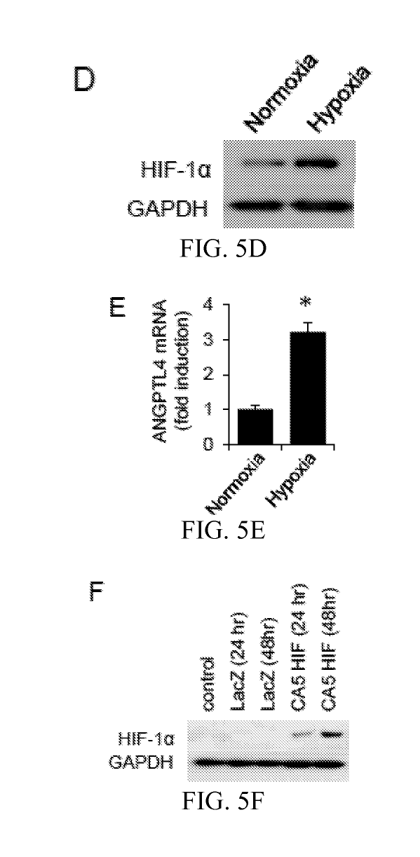

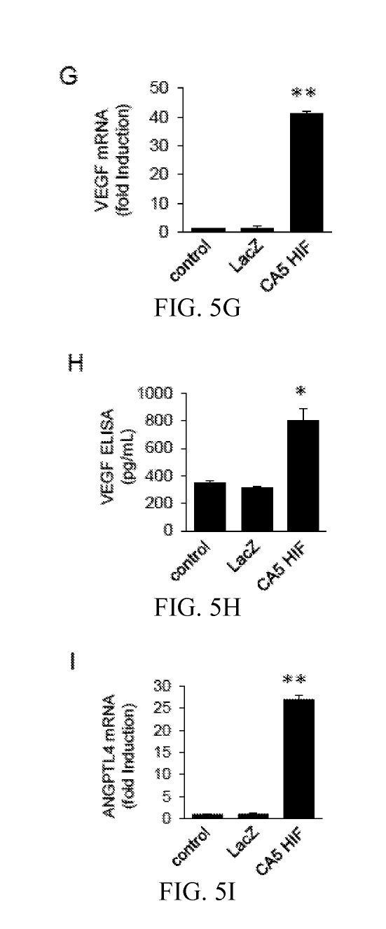

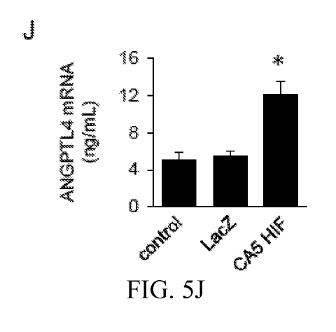

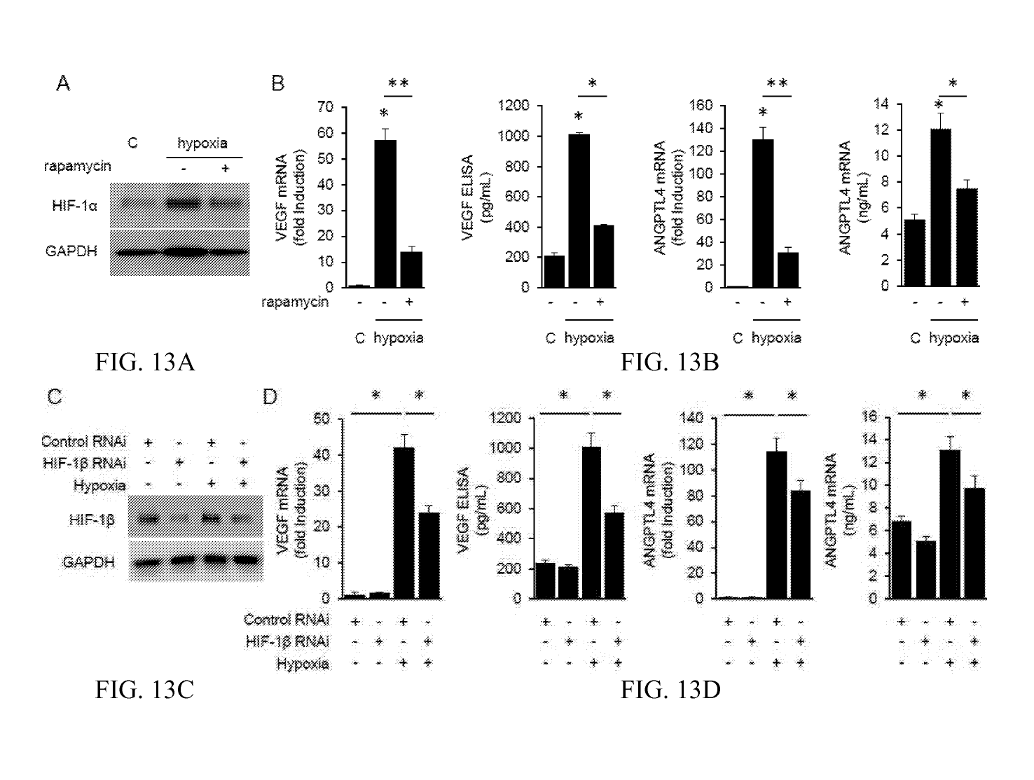

To address the relative contribution of other HIF-1-dependent secreted factors to the pathogenesis of DME, we analyzed changes in mRNA expression induced by exposure of MIO-M1 cells to hypoxia using an Affymetrix Gene Array with over 25,000 gene sequences. Transcripts of several known HIF-1 target genes that play key roles in cell survival (DDIT4 and HSP70-2), angiogenesis (VEGF and EDN1), metabolism (PFKFB4 and ALDOC), and pH regulation (CA9 and CA12) were upregulated in hypoxic MIO-M1 cells (Table 1). Expression of mRNA encoding the angiogenic cytokine, angiopoietin-like 4 (ANGPTL4) was also increased in hypoxic cells (19, 20). Indeed, of the over 25,000 genes screened in the hypoxia-treated MIO-M1 cells, ANGPTL4 was among the most highly induced genes (upregulated more than 9 fold). We confirmed that exposure of MIO-M1 cells to hypoxia induced ANGPTL4 mRNA and protein, and that ANGPTL4 mRNA was inhibited by digoxin and therefore is HIF-dependent (FIG. 5A-C). Similar results were observed in primary murine Müller cells (FIGS. 5D and E). These results were confirmed using a second HIF-inhibitor, rapamycin (FIG. 13A and B) and further corroborated using an RNAi approach targeting HIF-1β, thereby preventing the formation of the functional HIF-1 (or HIF-2) heterodimer (FIGS. 13C and D).

TABLE 1
Upregulation of Transcripts of HIF-1 Target Genes

| HIF-Target Genes | Fold Induction |
|---|---|
| Survival | |
| DDIT4 | 4.2 |
| HSP70-2 | 4.1 |
| IGFBP3 | 1.8 |
| IGFBP2 | 1.2 |
| IGFBP1 | 1.1 |
| Angiogenesis | |
| ANGPTL4 | 9.3 |
| VEGFA | 3.7 |
| EDN1 | 3.0 |
| END2 | 1.1 |
| PEDF | 0.9 |
| ANGPT1 | 0.8 |
| TIE1 | 0.7 |
| ANGPT2 | 0.4 |
| Metabolism | |
| PFKFB4 | 3.2 |
| ALDO C | 2.5 |
| PFKFB3 | 1.6 |
| ENO1 | 1.1 |
| LDHA | 1.1 |
| PKM2 | 1.0 |
| PDK | 0.8 |
| pH Regulation | |
| CA9 | 3.1 |
| CA12 | 3.0 |
| TGM2 | 1.3 |

ANGPTL4 has previously been shown to be upregulated by hypoxic stabilization of HIF (21-25). To confirm that stabilization of HIF-1α was sufficient to promote ANGPTL4 expression in retinal Müller cells, we infected MIO-M1 cells with AdCA5, a recombinant adenovirus expressing a constitutively active HIF-1α mutant (26). Infection of MIO-M1 cells with AdCA5 demonstrated that forced HIF-1α expression was sufficient to increase ANGPTL4 mRNA levels and protein secretion in non-hypoxic cells (FIG. 5F-J).

### EXAMPLE 6

### ANGPTL4 is Upregulated by HIF-1 in the Hypoxic Inner Retina In Vivo

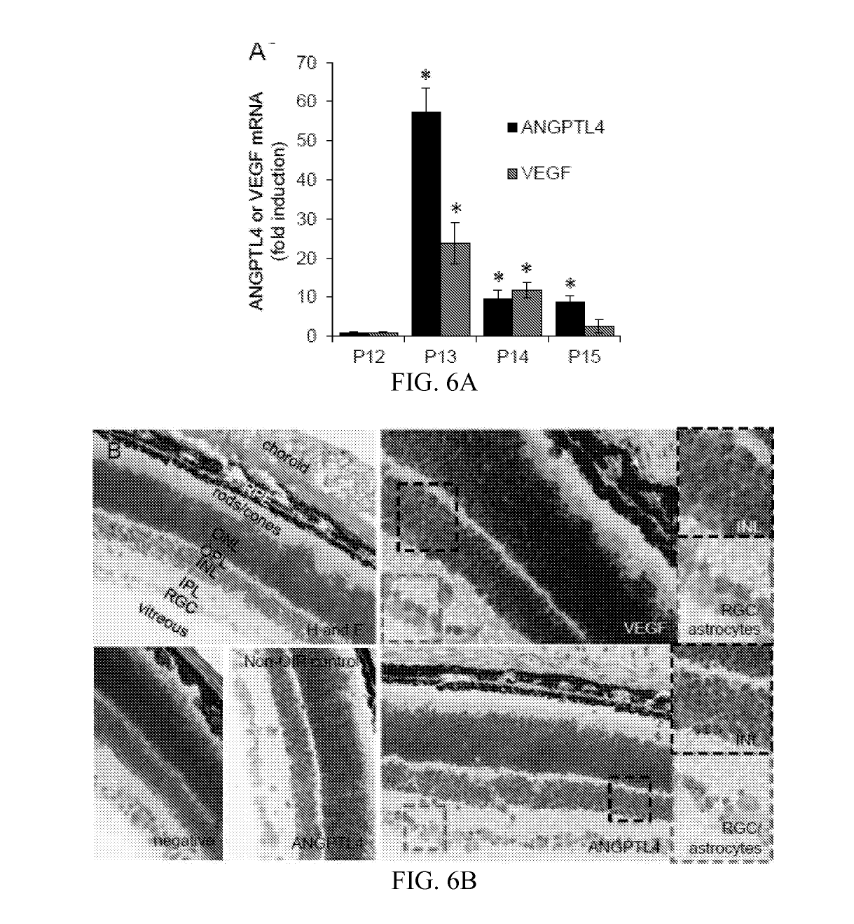

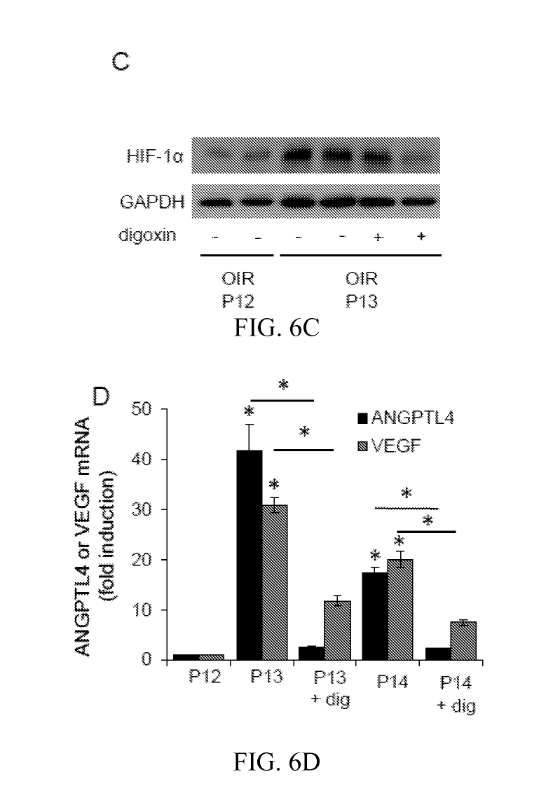

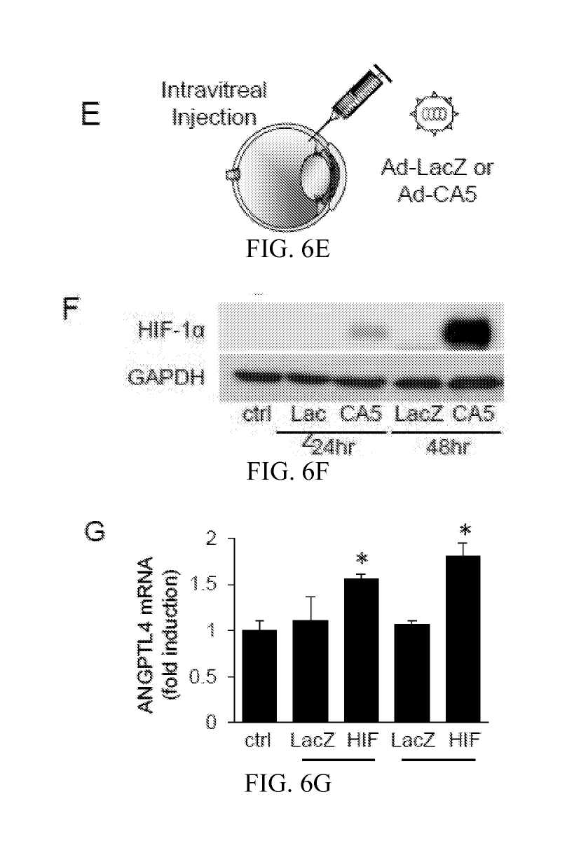

We next returned to the OIR model to examine the induction of ANGPTL4 in the ischemic retina in vivo and observed that Angptl4 mRNA was induced more than 50 fold in the ischemic retina—twice the effect seen with Vegf (paralleling the results observed in vitro)—and that the upregulation of Angptl4 was sustained for 72 hours following ischemia (FIG. 6A) Immunohistochemical analysis of eyes from OIR mice demonstrated strong expression of ANGPTL4 in the inner retinal layers in the posterior retina in these animals, similar to the expression pattern observed for VEGF (FIG. 6B). Only light expression was ANGPTL4 was detected in age-matched non-OIR mice Inhibition of HIF-1α protein accumulation in the ischemic inner retina with daily treatment with digoxin completely abolished the induction of Angptl4, but only partially inhibited the induction of Vegf (FIGS. 6C and D).

We next examined whether forced HIF-1α expression in the non-ischemic retina was sufficient to promote an increase in Angptl4 transcription in mice. Intravitreal injection of AdCA5 (FIG. 6E) resulted in an accumulation of stable HIF-1α protein within 48 hours (FIG. 6F) and an increase of Angptl4 mRNA by almost 2 fold (FIG. 6G).

### EXAMPLE 7

### ANGPTL4 Promotes Vascular Permeability In Vitro and In Vivo

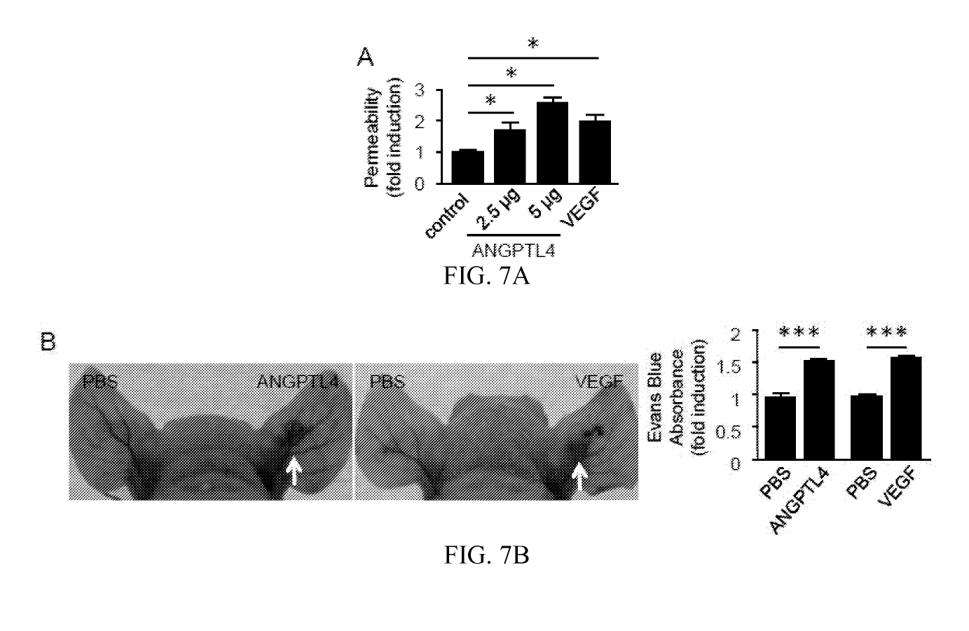

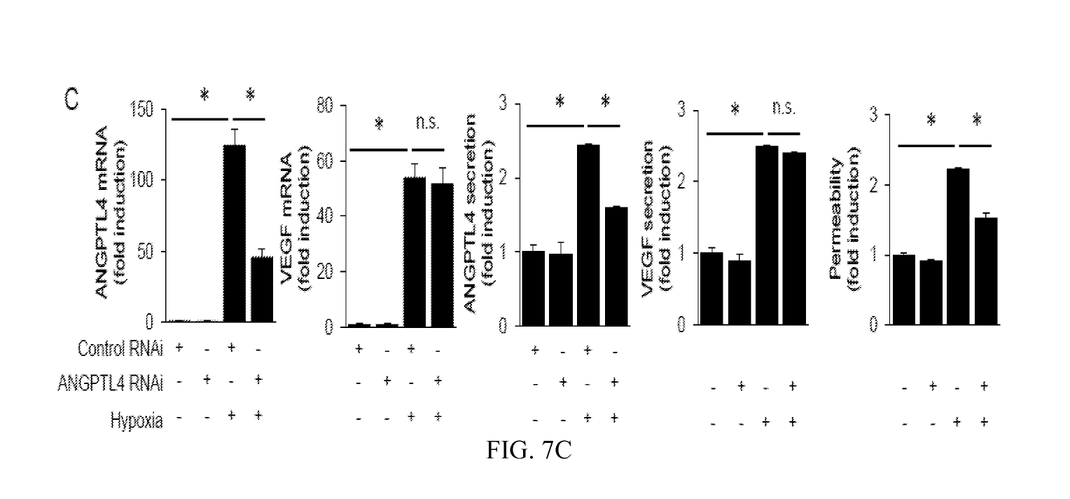

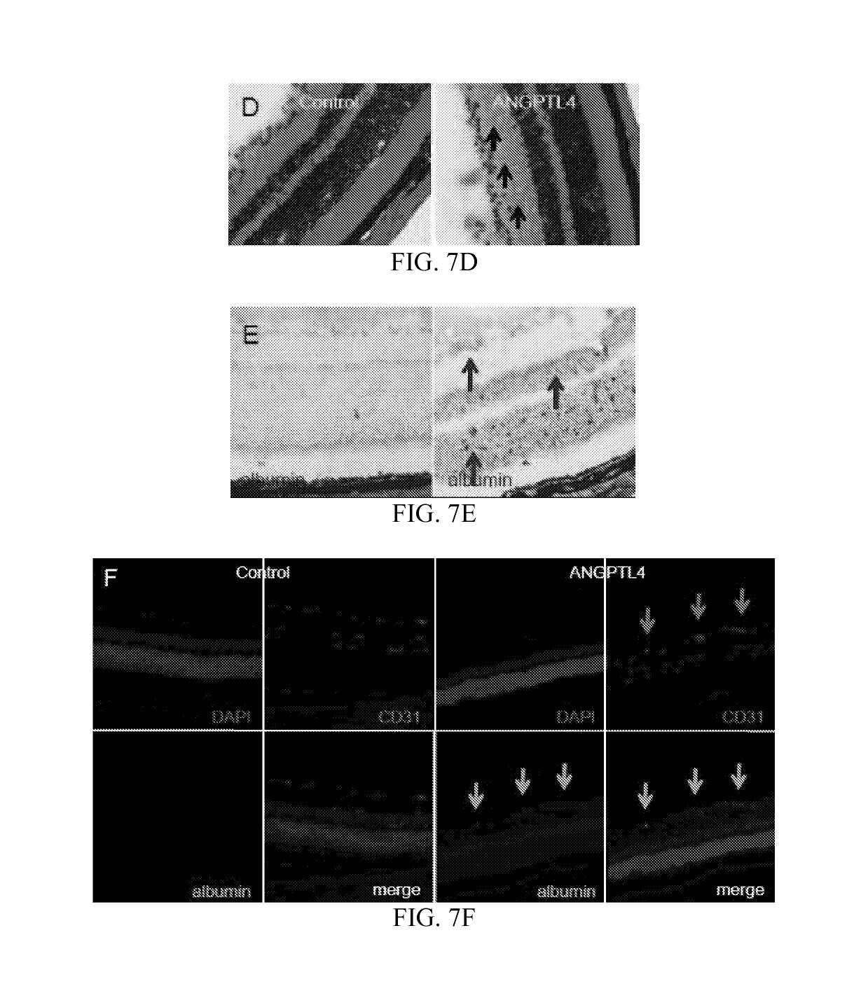

The role of ANGPTL4 in endothelial cell function remains controversial, and possibly tissue-specific (20). To directly assess whether ANGPTL4 was sufficient to promote vascular permeability in vitro, we treated monolayers of HMVECs with recombinant human ANGPTL4 and assessed the promotion of endothelial cell permeability as determined by passage of FITC-dextran. ANGPTL4 potently induced endothelial cell permeability, similar to VEGF (FIG. 7A). We next assessed whether ANGPLT4 could promote vascular permeability in vivo. To this end, we used the modified Miles assay to measure vascular permeability in the mouse ear following intradermal injection with PBS or ANGPTL4. We observed a remarkable increase in vascular permeability following treatment with ANGPTL4, similar to that observed with VEGF (FIG. 7B).

We next examined the potential contribution of ANGPTL4 to the promotion of vascular permeability by hypoxic Müller cells. To this end, we used RNA interference (RNAi) to specifically inhibit the expression of ANGPTL4. We treated monolayers of HMVECs with conditioned medium from hypoxic MIO-M1 cells pre-treated with ANGPTL4 RNAi Inhibition of ANGPTL4 significantly reduced the endothelial cell permeability promoted by hypoxic Müller cells (FIG. 7C).

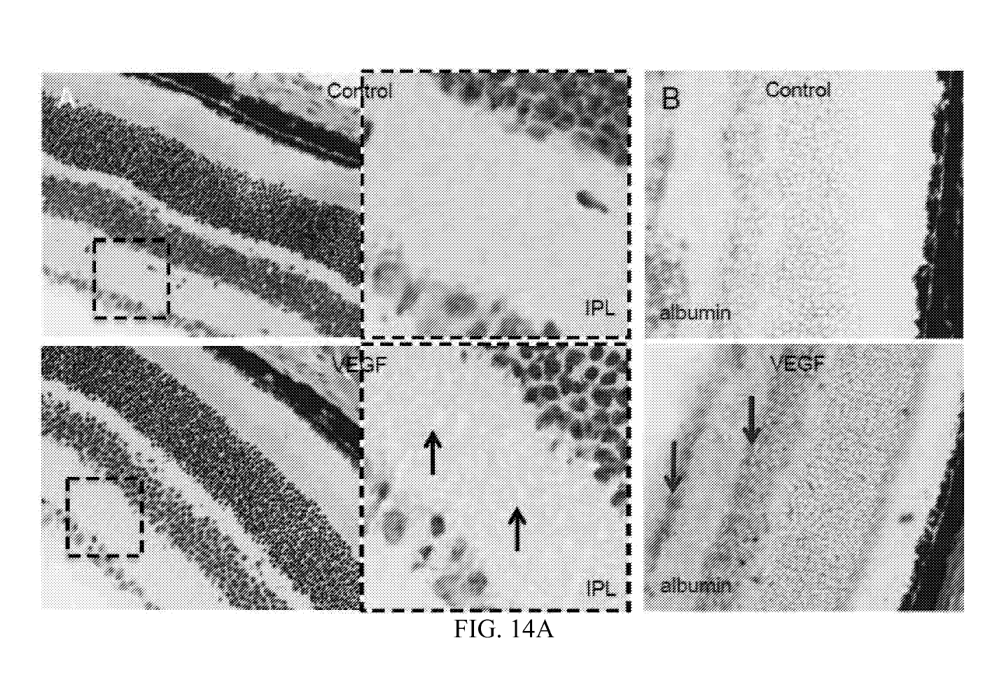

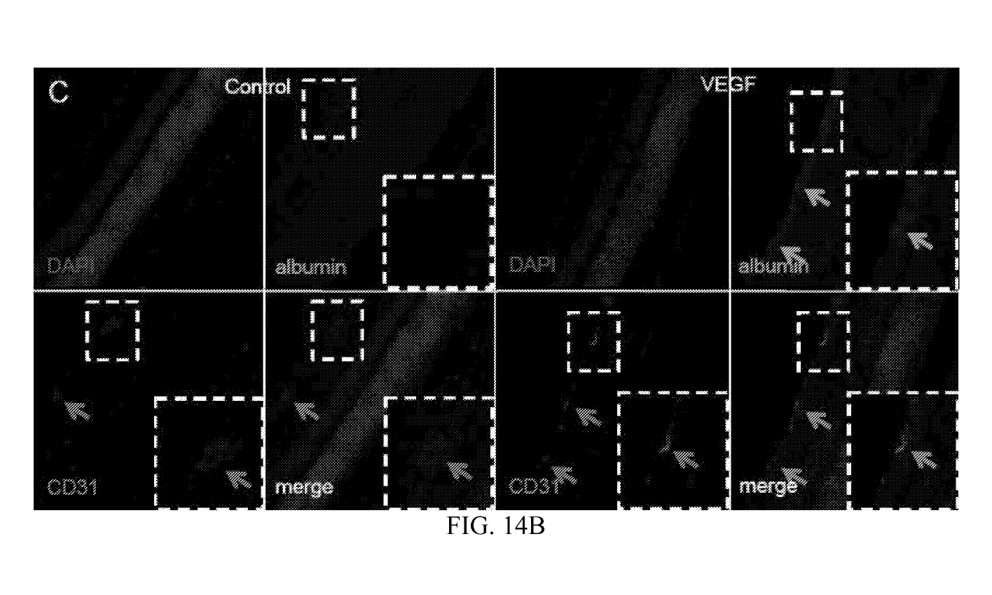

To directly assess whether ANGPTL4 was sufficient to promote vascular permeability in vivo, we injected recombinant murine (rm) ANGPTL4 intravitreally into the mouse eye and examined the retinas after 48 hours. We observed a marked and statistically significant increase in vascular permeability, resulting in leakage of plasma (FIG. 7D) and albumin (FIGS. 7E and F) into the interstitial tissue, similar to that seen with intravitreal injections with rmVEGF (FIGS. 14 and 15). Taken together, these results strongly support a role for ANGPTL4 in the promotion of vascular permeability in ischemic retinal disease.

### EXAMPLE 8

### ANGPTL4 is Expressed in the Inner Retina of Patients with Diabetic Eye Disease

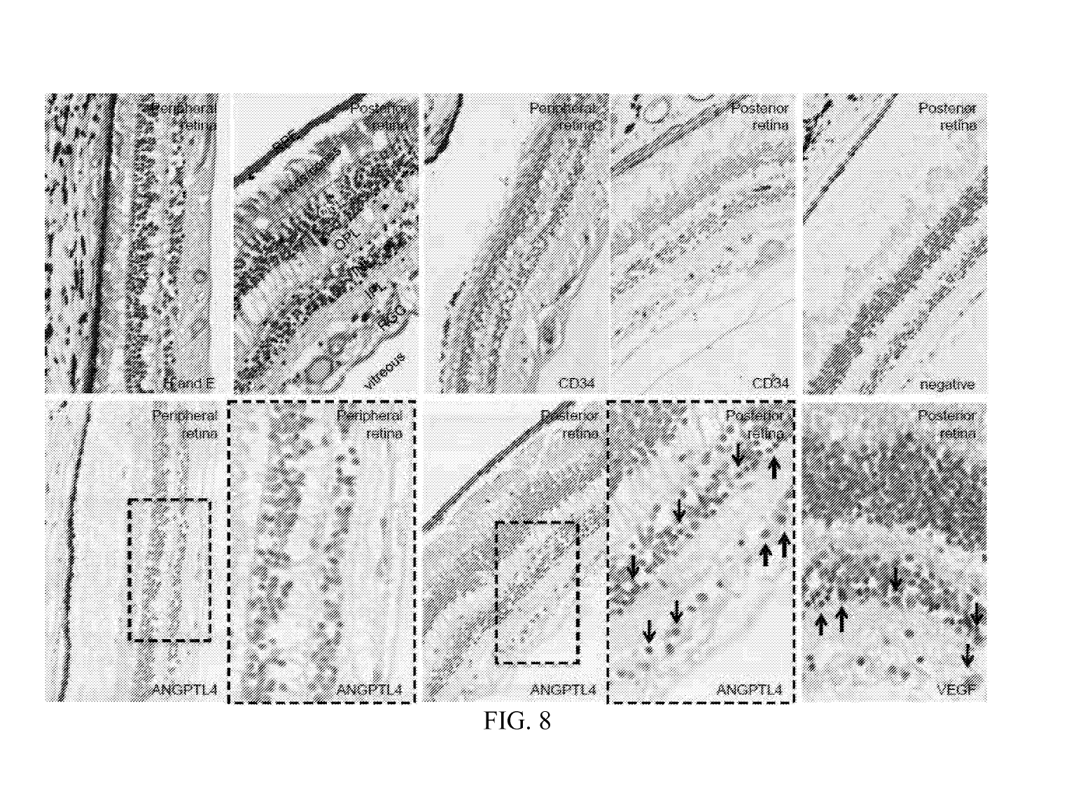

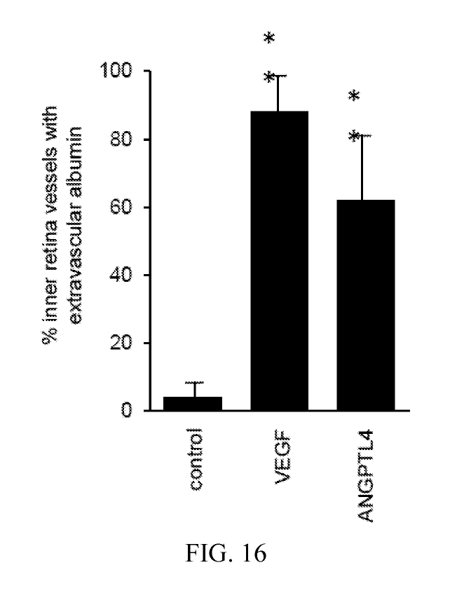

Examination of retinal tissue from 5 individuals with known DME revealed that ANGPTL4 was consistently expressed in the posterior ischemic inner retina, adjacent to areas of retinal edema, but was not observed in the peripheral (perfused) retina in all tissues examined (FIG. 8). This expression pattern was similar to that observed for VEGF, and was identical to the expression pattern of HIF-1α in ischemic (injured) Müller cells (FIG. 2H). Expression of ANGPTL4 was not detected in retinal tissue from normal (age-matched) control patients without a known diagnosis of an ischemic retinopathy (FIG. 16).

### Discussion

By 2050 the prevalence of diabetes will more than triple globally, dramatically increasing the burden of this disease worldwide (27). This will result in a concurrent rise in the number of patients with vision impairment from DME, the most common cause of severe vision loss in the working-age population in the developed world (9). Despite the recent introduction of therapies targeting VEGF, the majority of patients with DME do not respond with a significant gain in vision (11). An alternative approach for those patients who fail current anti-VEGF agents is to design treatment modalities that more efficiently inhibit VEGF; however, these efforts may have unwanted consequences. VEGF has been shown to play an important role as a neurotrophic factor and long-term inhibition of VEGF may potentially damage the neurosensory retina (28, 29). The observation that loss of a single copy of Vegf is embryonic lethal in mice demonstrates the importance of this potent growth factor in development (30). Collectively, these considerations support the rationale for the identification and targeting of other factors that participate in the pathogenesis of DME.

We provide evidence here that HIF-1 is a target for the treatment of macular edema in ischemic retinopathies. In animal models of ischemic retinopathies, inhibition of HIF-1α has been previously shown to also prevent retinal neovascularization (31). These observations argue in favor of therapies directed against HIF-1 as a broad-spectrum approach to target multiple hypoxia-inducible genes that promote vascular permeability. However, HIF-1 plays a fundamental role in acclimating cells to ischemia: HIF-1 regulates the metabolic shift from respiration to aerobic glycolysis and lactic acid production; stimulates nutrient supply by influencing adaptive survival mechanisms (e.g., autophagy and lipid and glycogen synthesis and storage); protects cells from oxidative stress; and safeguards cells from acidosis (32, 33). In concert with the angiogenic genes regulated by HIF-1, the responsible genes work together to collectively promote the survival of cells/tissue exposed to chronic ischemia. Inhibition of HIF-1 may therefore have undesirable effects on the highly metabolically-active retina. Further studies examining the sequelae of chronic HIF inhibition on the retina are necessary before this approach could be brought to the clinic.

An alternative strategy for the treatment of patients with ischemic retinopathies may be to instead identify and inhibit only the specific HIF-1-dependent target gene products that promote macular edema, as has been demonstrated by anti-VEGF therapies. In this regard, we demonstrate here that ANGPTL4 is a key HIF target gene expressed in the inner retina by hypoxic Müller cells that promotes vascular permeability in ischemic retinal disease. Angiopoietins have been described as critical factors in vascular development (34). Angiopoietin-1 (ANGPT-1) promotes vessel maturation, whereas angiopoietin-2 (ANGPT-2) antagonizes its effect on vessel stabilization. ANGPTL4 is a secreted glycoprotein that—unlike ANGPT-1 and ANGPT-2—does not bind to the TIE-2 receptor, and remains an orphan ligand (19, 20, 35). Although ANGPTL4 secretion has been shown to modulate the disposition of circulating triglycerides by inhibiting lipoprotein lipase (20, 36), its role in vascular biology is less clear.

Initial studies on the role of ANGPLT4 in cancer demonstrated that this cytokine may inhibit angiogenesis and tumor metastasis (37-41). However, more recent reports suggest that ANGPTL4 may be pro-angiogenic and pro-vascular permeability 23, 25, 42-46). ANGPTL4 has been shown to disrupt vascular endothelial cell-cell (tight and adherens) junctions, facilitating cellular trans-endothelial passage and tumor dissemination (47). ANGPTL4 has further been found to promote the angiogenic and exudative phenotypes characteristic of the unique vascular tumor, Kaposi's sarcoma, and to activate the Rho-ROCK pathway (48). More recently, it has been demonstrated that ANGPTL4 may promote the disruption of vascular integrity by directly interacting with integrin α5β1, vascular endothelial (VE)-cadherin, and claudin-5 in a temporally sequential manner (49).

In the context of the eye, this multifaceted cytokine has also been shown to be both pro- and anti-angiogenic (50, 51). Recently, examination of the eyes of homozygous Angptl4 null mice suggested that ANGPTL4 is pro-angiogenic (51). However, contrary to the observations described here, the authors of the work cited above also found that loss of expression of ANGPTL4 resulted in an increase in vascular permeability in the developing retina. These disparate results may be a consequence of compensatory changes in the levels of other factors that also affect vascular permeability (e.g., VEGF) during development of the retina in the Angptl4 null mice. It may also be that ANGPTL4 plays different roles during vascular development than it does in pathological angiogenesis in the eye.

In addition to VEGF, the list of cytokines and growth factors that have been proposed to participate in the pathogenesis of diabetic eye disease is long. This includes—but is not limited to—angiopoietins, interleukins, platelet derived growth factor, fibroblast growth factor, hepatocyte growth factor, transforming growth factor, placental endothelial cell growth factor, connective tissue growth factor, angiotensin, and monocyte chemotactic protein. However, to date, only VEGF has proven to be an effective target for the treatment of vascular hyper-permeability in DME. Our results suggest that, like VEGF, ANGPTL4 plays an important role in promoting vessel permeability in patients with ischemic retinopathies. We propose that ANGPTL4 is an important mediator of DME and our findings provide the foundation for inhibition of ANGPTL4 as a therapeutic approach for the treatment of this vision threatening disease.

### EXAMPLE 9

### RPE Cells Secrete ANGPTL4 to Promote Choroidal Neovascularization in Neovascular Age-Related Macular Degeneration

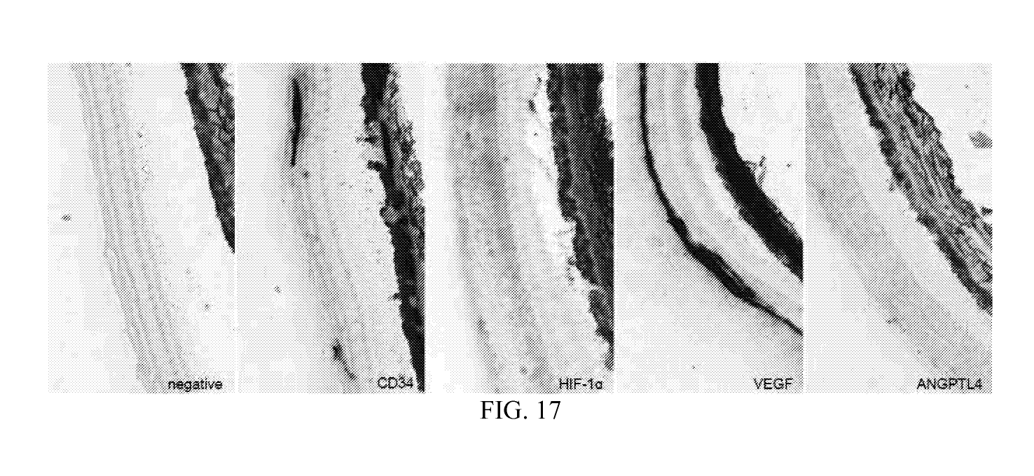

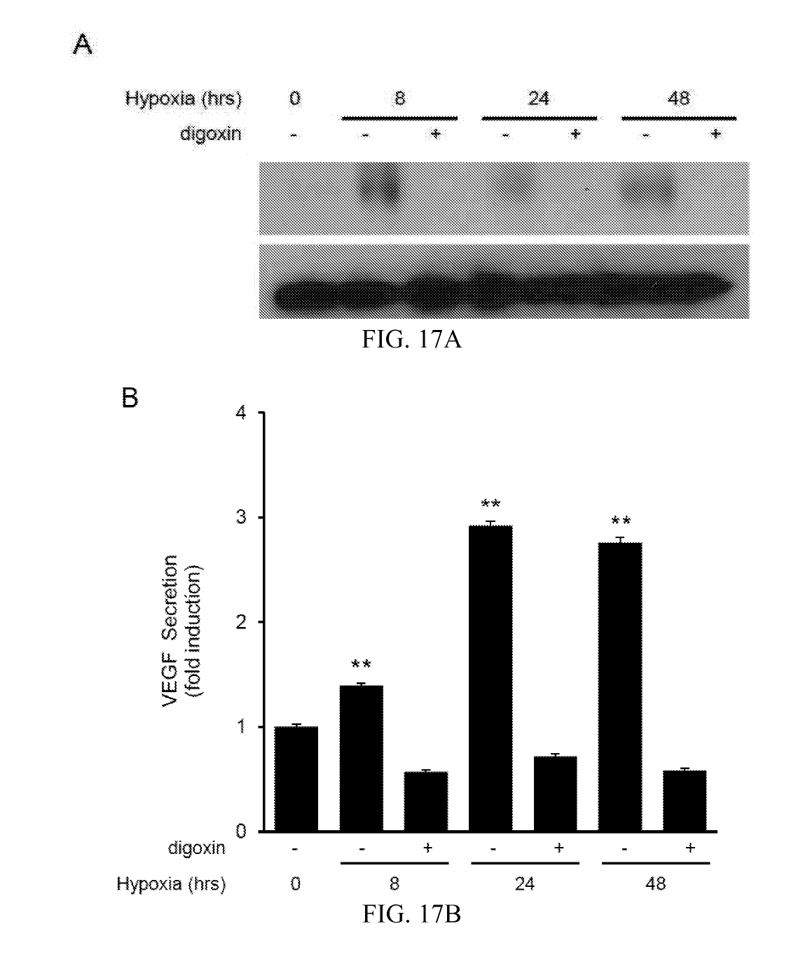

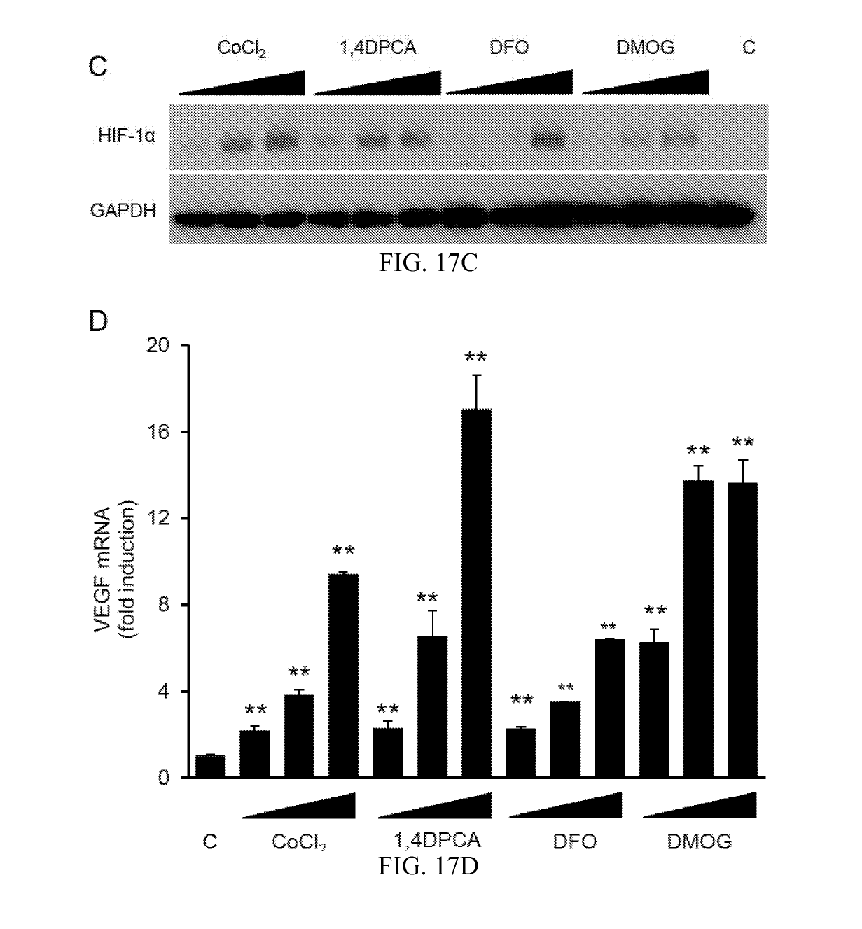

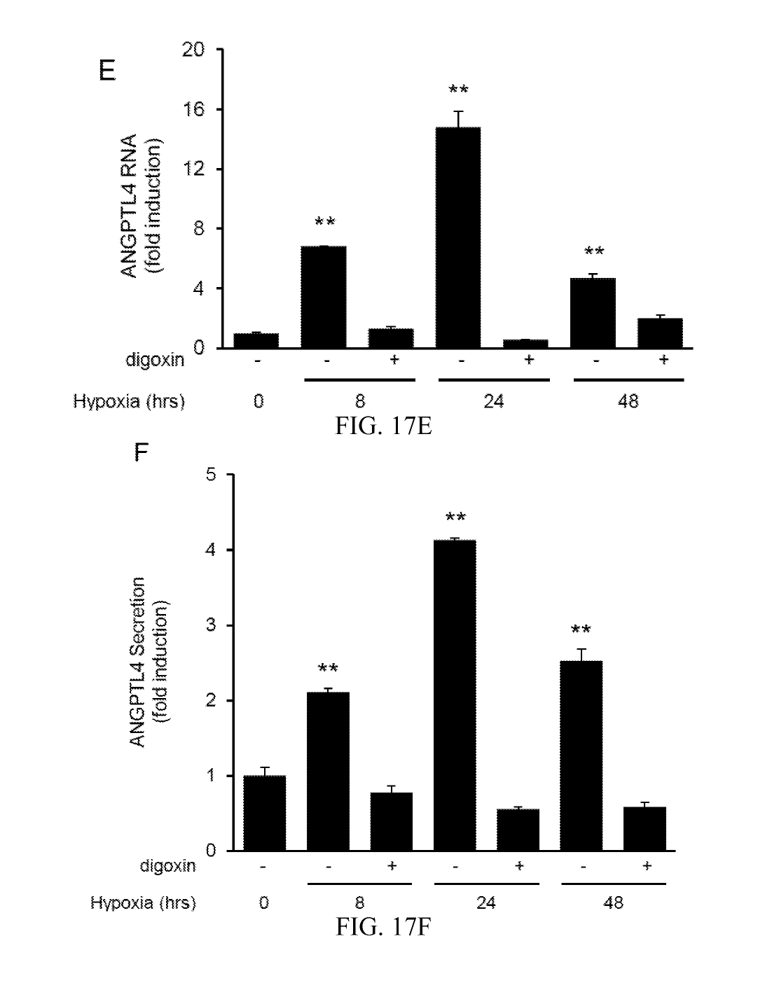

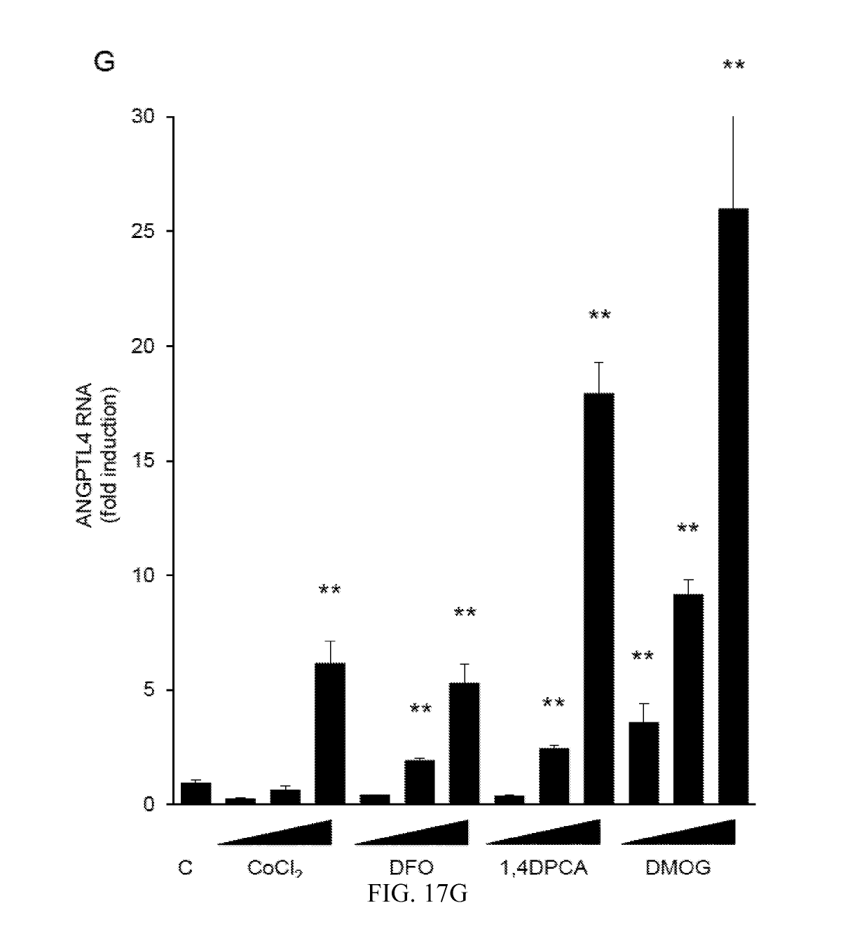

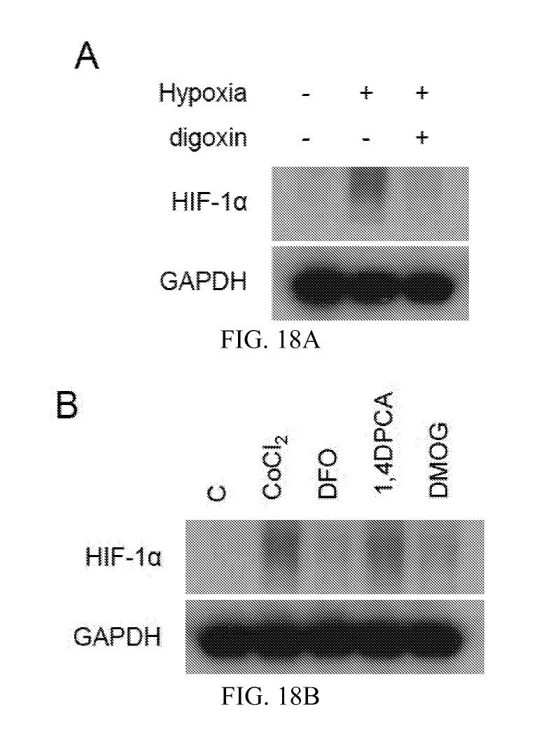

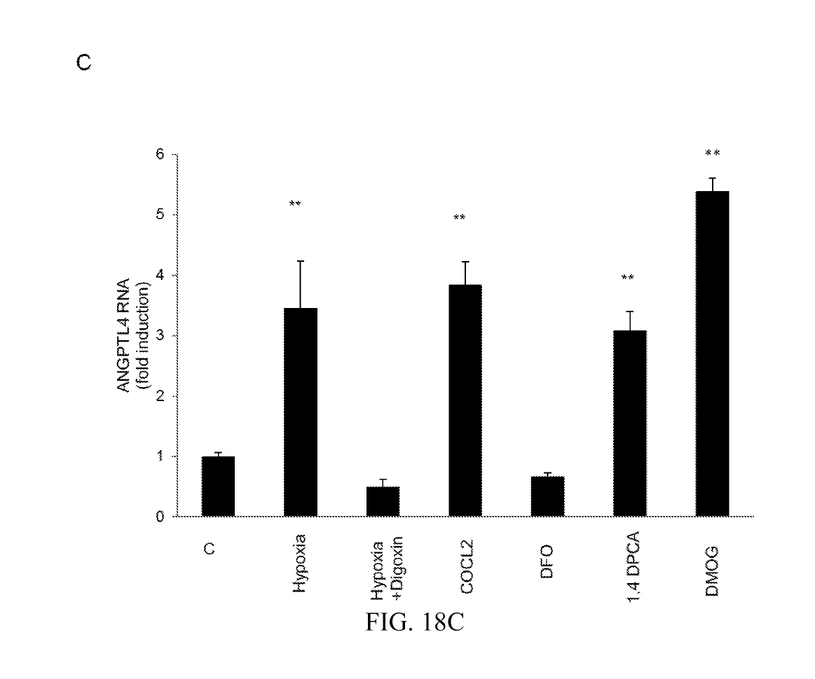

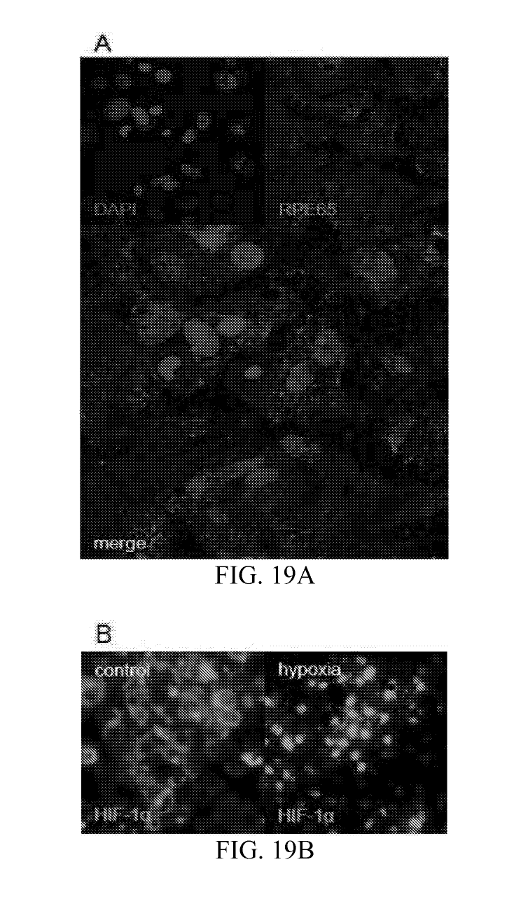

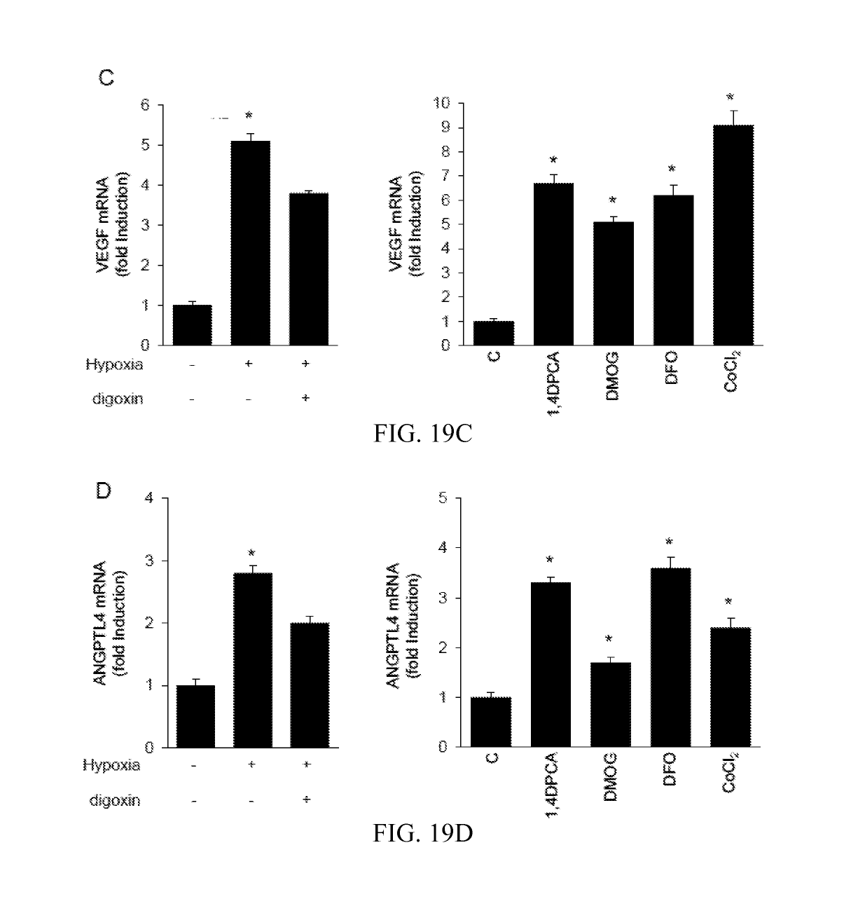

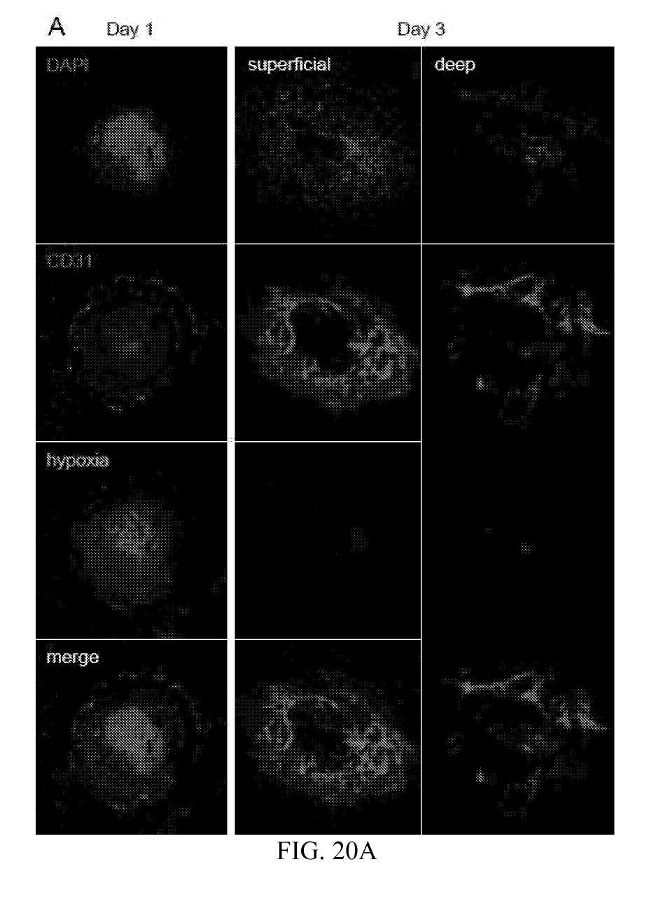

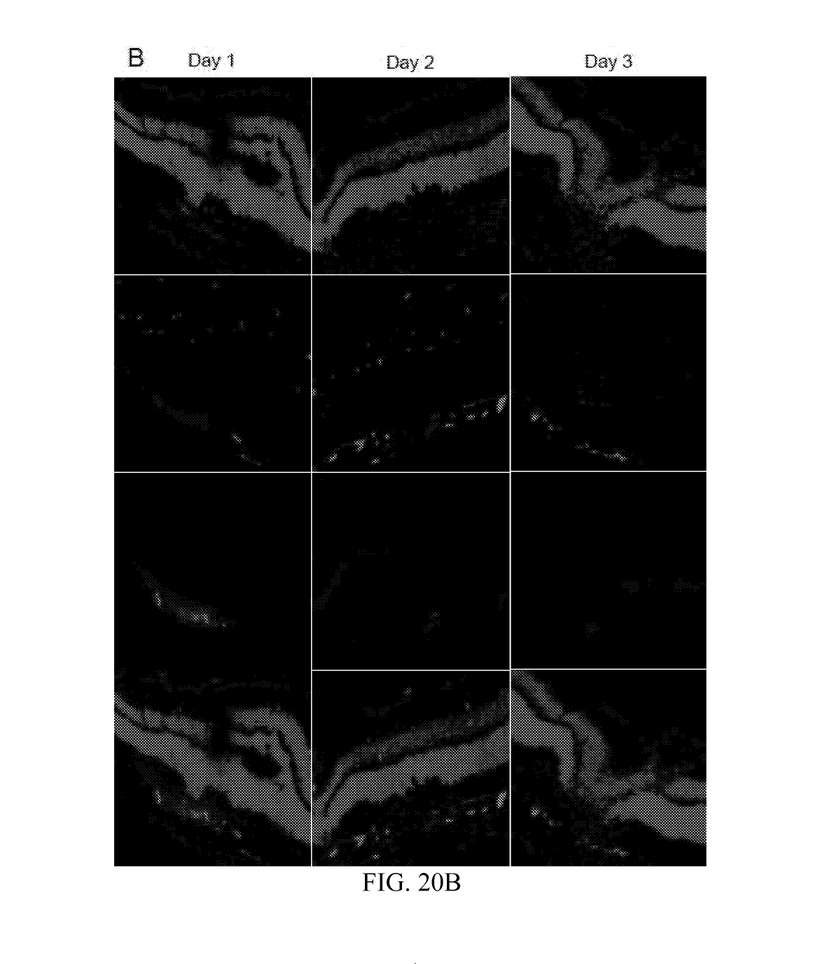

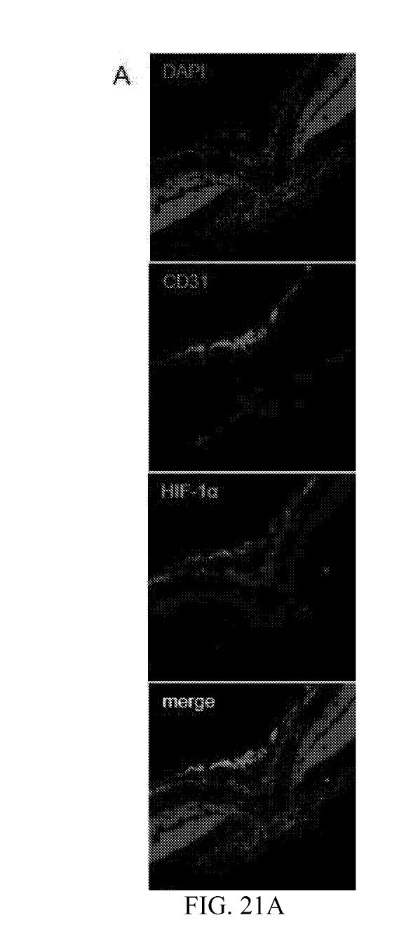

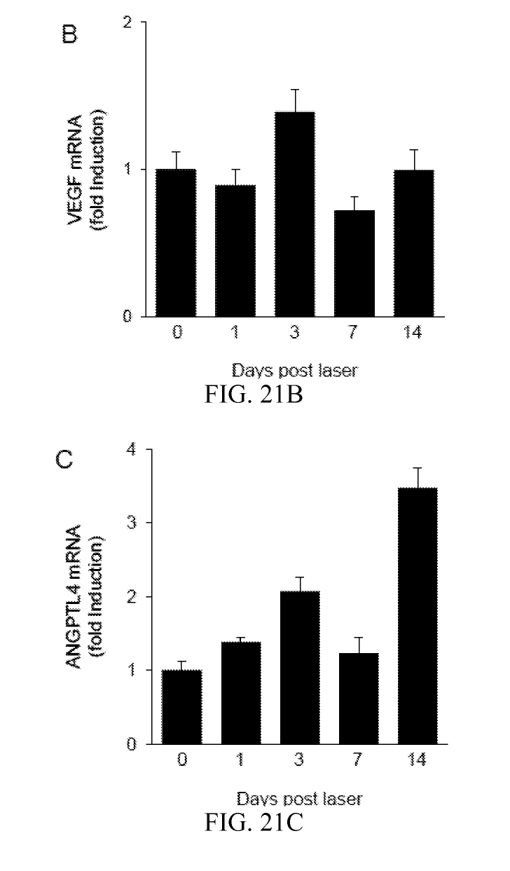

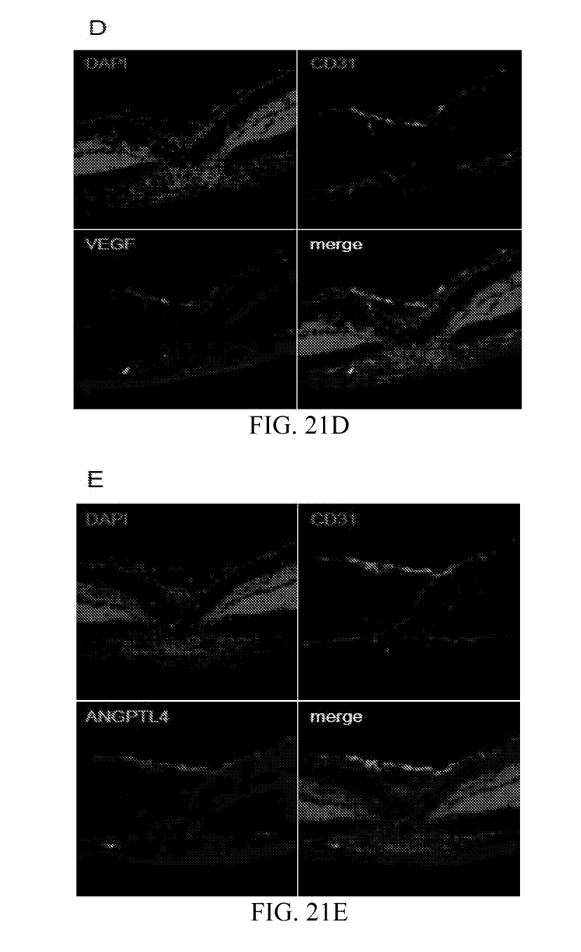

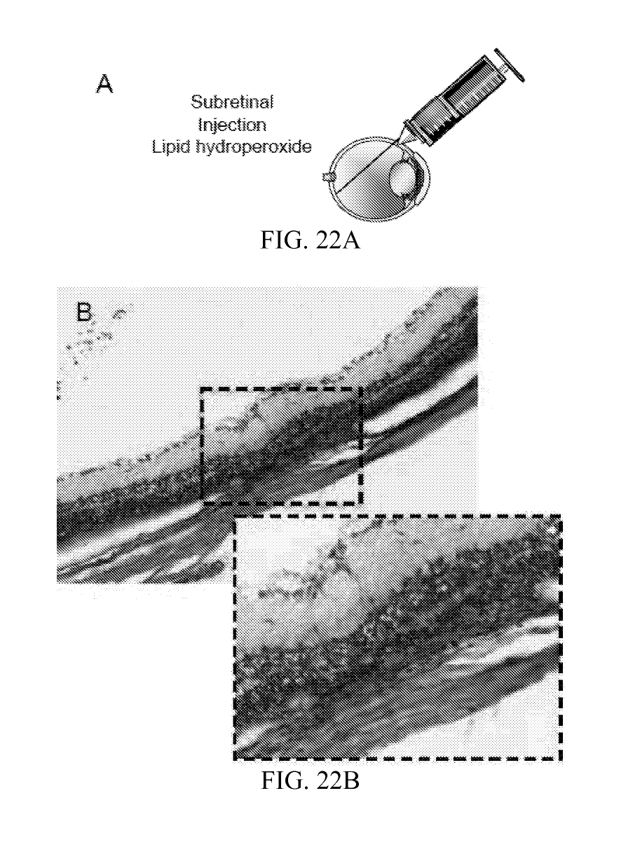

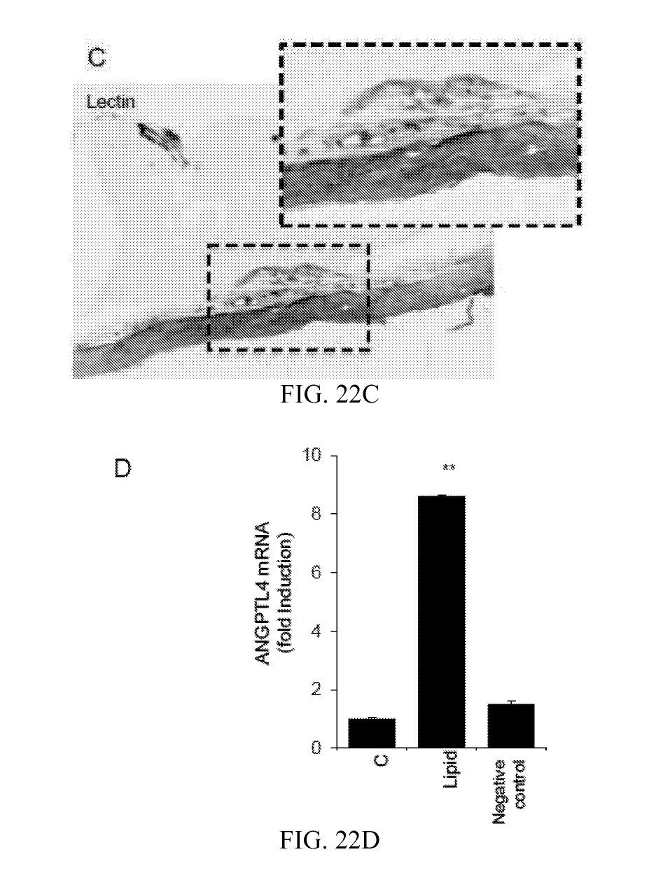

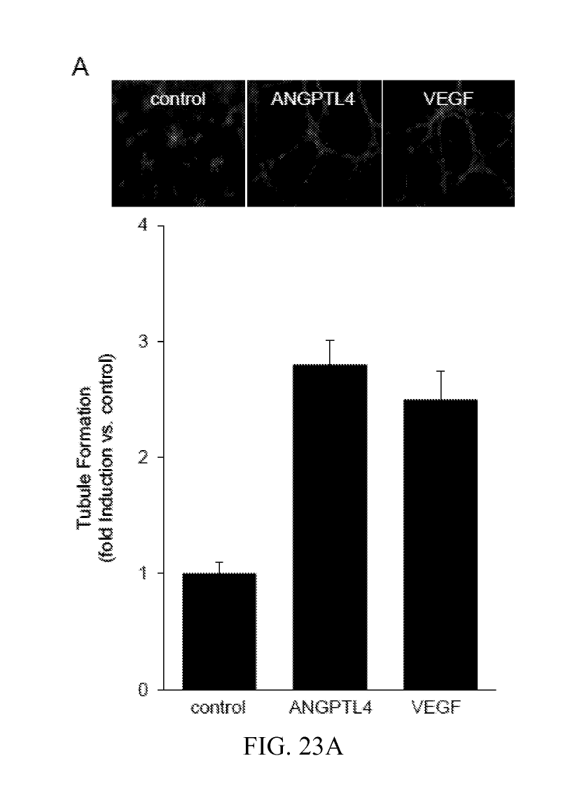

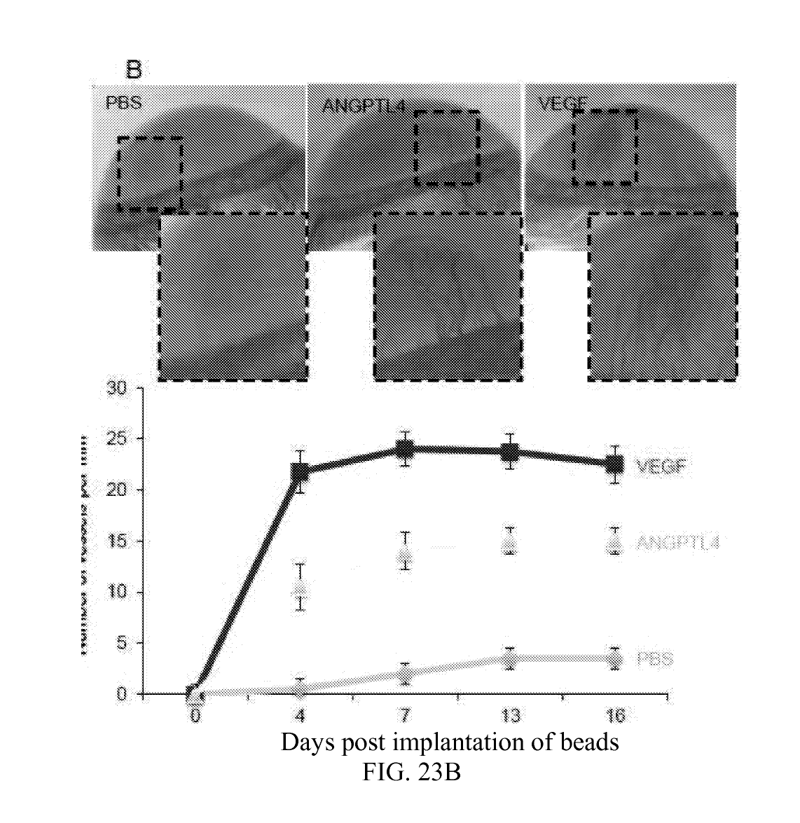

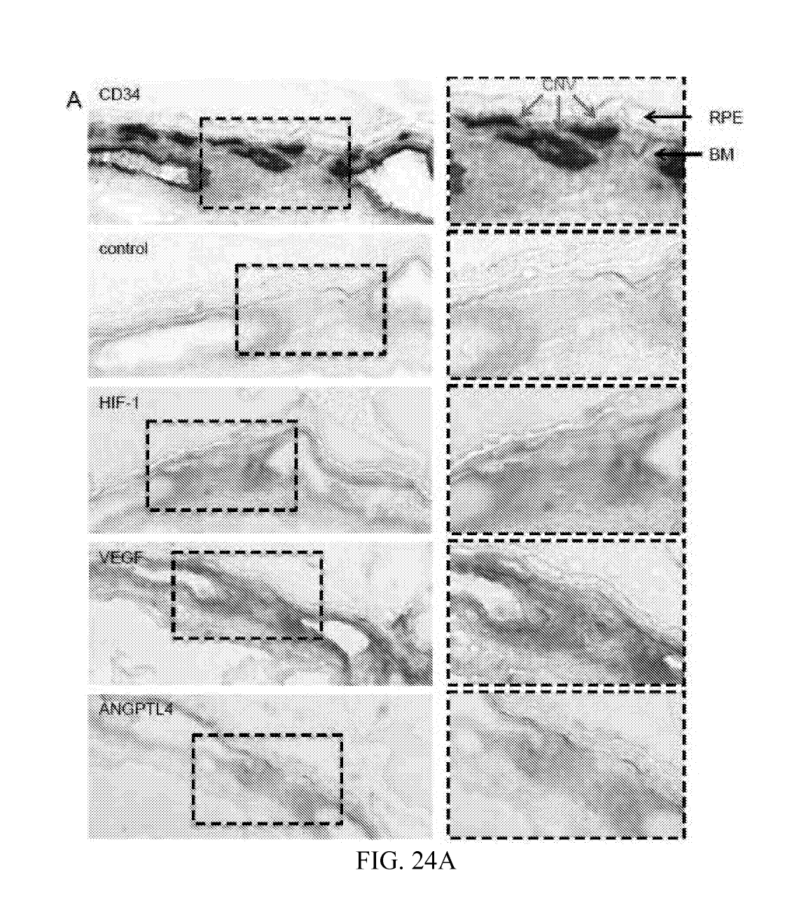

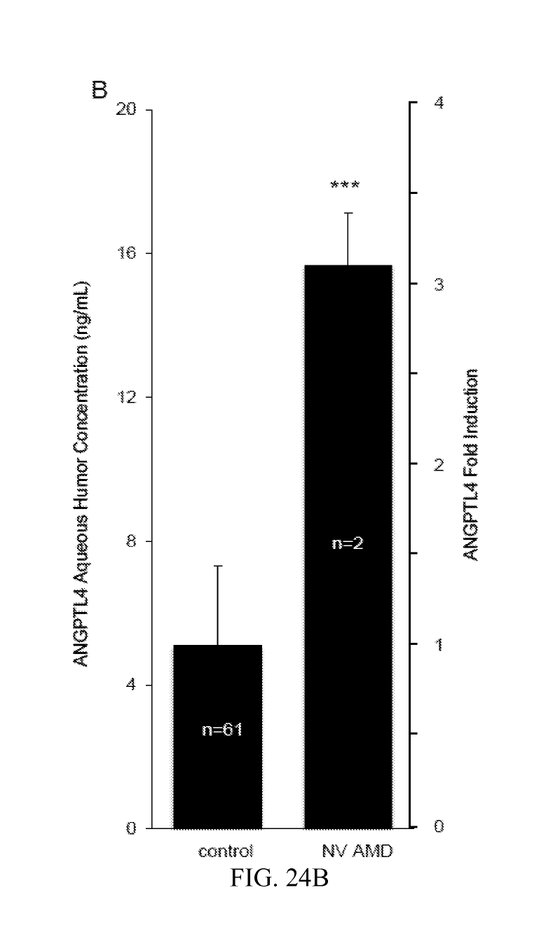

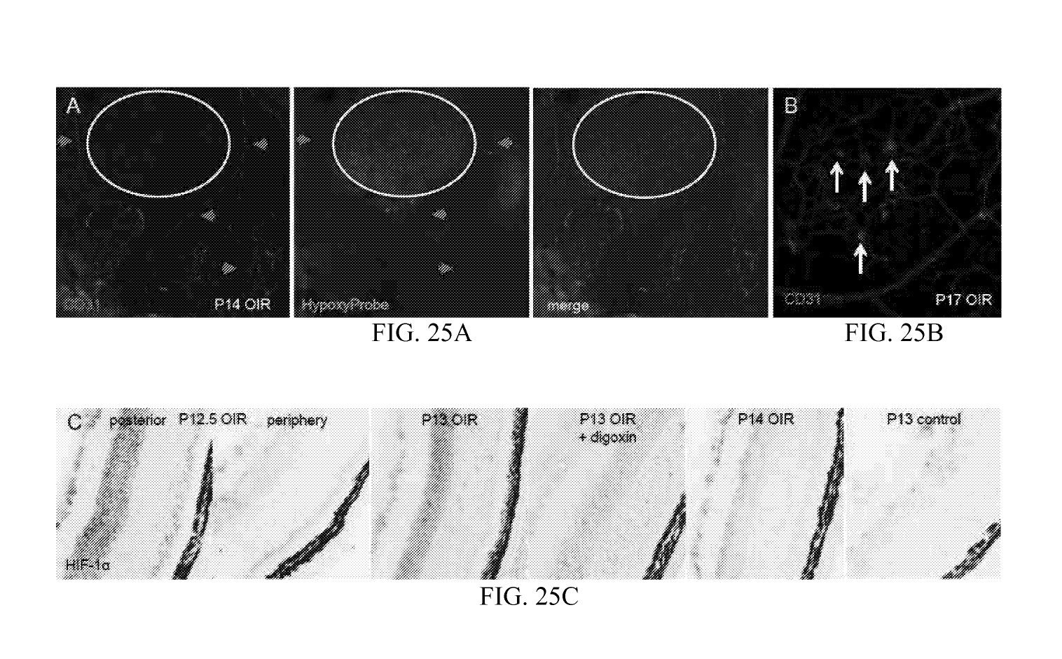

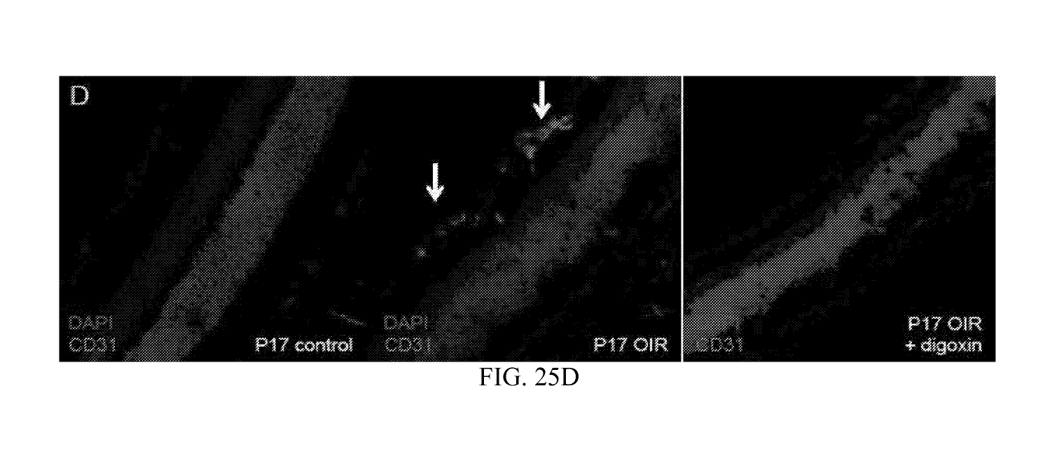

Cell Culture: ARPE19, human ES derived RPE cells, primary murine RPE cells. Rat: Subretinal lipid hydroperoxide CNV Model. Assays used included Cornea pocket assay, Western Blot and RT-PCR, and Immunohistochemistry and Immunofluorescence. Patient Samples: ELISA analysis of ANGPTL4 levels in aqueous humor. See FIGS. 17-25.

Age-related macular degeneration (AMD) is a complex and multifactorial illness and is the most common age-related disease causing blindness in industrialized countries. Neovascular (NV) or “wet” AMD is the most destructive form of AMD and it is characterized by choroidal neovascularization (CNV), the invasion of new blood vessels (pathological angiogenesis) into subretinal spaces. CNV membranes can result in exudation and bleeding, and forms (disciform) scars involving the fovea, resulting in irreversible loss of central vision.

The growth of pathological vessels in CNV is dependent on angiogenic mediators that are regulated by the transcriptional enhancer, hypoxia-inducible factor (HIF)-1. HIF-1 is a heterodimeric protein composed of an exquisitely oxygen-sensitive HIF-1α subunit and a ubiquitous HIF-1β subunit. Under normoxic conditions, specific proline residues on the HIF-1α subunit are hydroxylated by a family of HIF Prolyl Hydroxylases (PHDs). This allows binding of the von Hippel-Lindau protein (pVHL), resulting in ubiquitination and degradation of HIF-1α by the cellular proteasome machinery. An additional level of regulation is provided by an asparagyl hydroxylase, the Factor Inhibiting HIF (FIH). FIH hydroxylates a specific asparagine residue on HIF-1α and prevents binding of the transcriptional co-factor, p300, to HIF-1α, thereby inhibiting its transcriptional activity. However, under hypoxic conditions (or low pH, low glucose, inflammation, oxidative stress, etc.), the PHDs and FIH no longer hydroxylate HIF-1α. pVHL therefore cannot bind to HIF-1α, and its degradation is reduced; conversely, p300 is able to bind to HIF-1α, and its transcriptional activity is enhanced. The resulting increased amount of active HIF-1α protein localizes to the nucleus and binds to HIF-1β forming HIF-1α/β (HIF-1) heterodimers. In NV AMD, stabilized HIF-1α protein localizes to the nucleus and binds to HIF-1β forming a heterodimer (HIF-1) that binds to the DNA of specific angiogenic genes. The most critical of the HIF-1-dependent secreted factors in NV AMD is vascular endothelial growth factor (VEGF). Indeed, the introduction of therapies that target VEGF has had a remarkable impact on the treatment of NV AMD. However, results from clinical trials using anti-VEGF therapies have demonstrated a significant improvement in vision (defined as a gain of at least 15 letters—or three lines—on the ETDRS vision chart) in a minority of patients. This underscores the need for the identification of other factors that contribute to NV AMD.

In this regard, we have recently identified angiopoietin-like 4 (ANGPTL4) as a novel HIF-dependent cytokine potently upregulated by HIF-1 in hypoxic Müller cells that is both necessary and sufficient for the promotion of vessel permeability in diabetic macular edema. Here, we provide evidence that stabilization of HIF-1α in RPE cells promotes the transcription and secretion of ANGPTL4 in NV AMD. ANGPTL4 promoted angiogenesis both in vitro and in vivo. ANGPTL4 is an essential therapeutic target for the treatment of patients with NV AMD.

### EXAMPLE 10

### ANGPTL4 in Proliferative Diabetic Retinopathy (PDR)

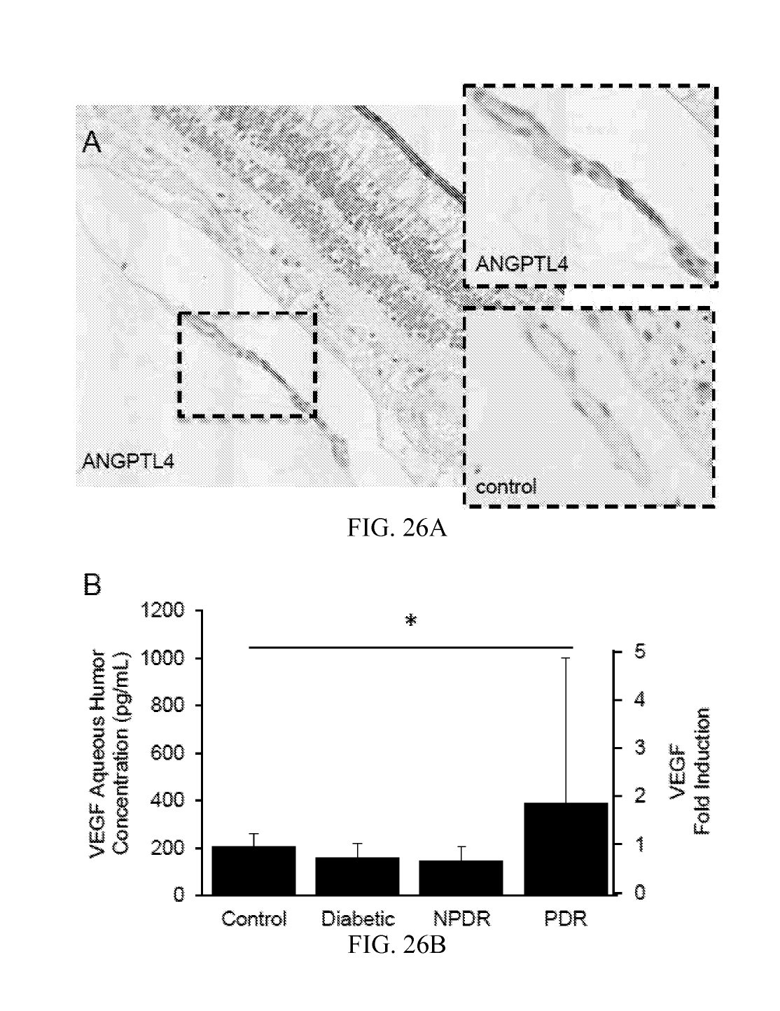

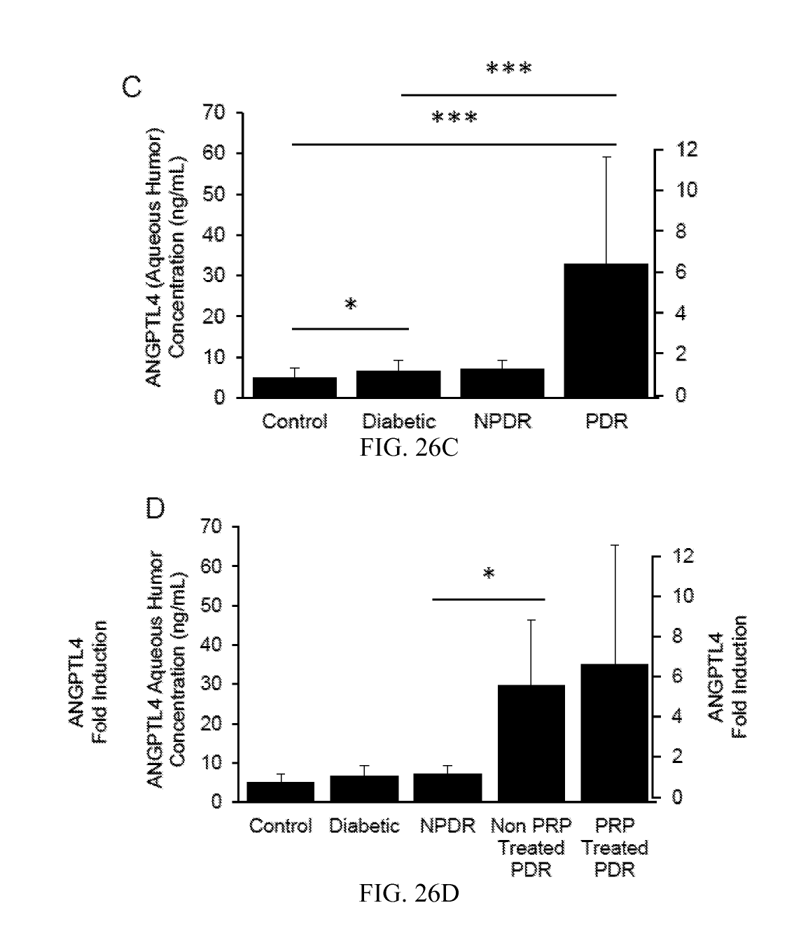

See FIG. 26.

TABLE 2
Patient Information on VEGF for PDR

| VEGF | Controls (n = 56^{a}) | DM, No DR (n = 17) | DM, NPDR (n = 3) | DM, PDR (n = 27^{b}) |
|---|---|---|---|---|
| Age (range) | 66.9 (41-91) | 69.2 (54-91) | 77.7 (65-96) | 51.0 (31-76) |
| Gender (M:F) | 17:34 | 6:11 | 0:3 | 12:8 |
| CVD^{c} | 25 | 10 | 3 | 15 |

^{a}includes 1 patient who had an aqueous sample taken from the same eye twice and 4 patients who had an aqueous sample taken from each eye
^{b}includes 3 patients who had an aqueous sample taken from the same eye twice, 2 patients who had an aqueous sample taken from each eye, and one patient who had an aqueous sample taken from the same eye twice and from the other eye once
^{c}includes any patient with a known history of hypertension, hypercholesterolemia, coronary artery disease, or cerebral vascular accident

TABLE 3
Patient Information on ANGPTL4 for PDR

| ANGPTL4 | Controls (n = 61^{a}) | DM, No DR (n = 21^{b}) | DM, NPDR (n = 3) | DM, PDR (n = 27^{c}) |
|---|---|---|---|---|
| Age (range) | 67.6 (41-91) | 69.1 (54-91) | 77.7 (65-96) | 51.0 (31-76) |
| Gender (M:F) | 19:37 | 6:14 | 0:3 | 12:8 |
| CVD^{d} | 27 | 13 | 3 | 15 |

^{a}includes 1 patient who had an aqueous sample taken from the same eye twice and 4 patients who had an aqueous sample taken from each eye
^{b}includes 1 patient who had an aqueous sample taken from each eye
^{c}includes 3 patients who had an aqueous sample taken from the same eye twice, 2 patients who had an aqueous sample taken from each eye, and one patient who had an aqueous sample taken from the same eye twice and from the other eye once
^{d}includes any patient with a known history of hypertension, hypercholesterolemia, coronary artery disease, or cerebral vascular accident

### References

1. Bek T (2009) Inner retinal ischaemia: current understanding and needs for further investigations. Acta Ophthalmol 87(4):362-367.

2. Scholl S, Augustin A, Loewenstein A, Rizzo S, & Kupperman B (2010) General pathophysiology of macular edema. (Translated from Eng) Eur J Ophthalmol 21(S6):10-19 (in Eng).

3. Crawford T N, Alfaro D V, 3rd, Kerrison J B, & Jablon E P (2009) Diabetic retinopathy and angiogenesis. Curr Diabetes Rev 5(1):8-13.

4. Michaelson I C (1948) The mode of development of the vascular system of the retina with some observations on its significance for certain retinal disorders. Trans. Ophthalmol. Soc. UK 68:137-180.

5. Maxwell PH (2005) Hypoxia-inducible factor as a physiological regulator. Exp Physiol 90(6):791-797.

6. Schofield C J & Ratcliffe P J (2004) Oxygen sensing by HIF hydroxylases. Nat

Rev Mol Cell Biol 5(5):343-354.

7. Semenza G L (2004) Hydroxylation of HIF-1: oxygen sensing at the molecular level. Physiology (Bethesda) 19:176-182.

8. Semenza G L (2012) Hypoxia-inducible factors in physiology and medicine. Cell 148(3):399-408.

9. Lang G E (2012) Diabetic macular edema. Ophthalmologica 227 Suppl 1:21-29.

10. Nagy J A, Benjamin L, Zeng H, Dvorak A M, & Dvorak H F (2008) Vascular permeability, vascular hyperpermeability and angiogenesis. Angiogenesis 11(2): 109-119.

11. Javey G, Schwartz S G, & Flynn H W, Jr. (2012) Emerging pharmacotherapies for diabetic macular edema. Exp Diabetes Res 2012:548732.

12. Witkin A J & Brown G C (2011) Update on nonsurgical therapy for diabetic macular edema. Curr Opin Ophthalmol 22(3):185-189.

13. Smith L E, et al. (1994) Oxygen-induced retinopathy in the mouse. Invest Ophthalmol Vis Sci 35(1):101-111.

14. Montezuma S R, Vavvas D, & Miller J W (2009) Review of the ocular angiogenesis animal models. Semin Ophthalmol 24(2):52-61.

15. Mowat F M, et al. (2010) HIF-1alpha and HIF-2alpha are differentially activated in distinct cell populations in retinal ischaemia. PLoS One 5(6):e11103.

16. Xing C, Hayakawa K, Lok J, Arai K, & Lo E H (2012) Injury and repair in the neurovascular unit. Neurol Res 34(4):325-330.

17. Zhang H, et al. (2008) Digoxin and other cardiac glycosides inhibit HIF-1 alpha synthesis and block tumor growth. Proc Natl Acad Sci USA 105(50):19579-19586.

18. Limb G A, Salt T E, Munro P M, Moss S E, & Khaw P T (2002) In vitro characterization of a spontaneously immortalized human Muller cell line (MIO-M1). Invest Ophthalmol Vis Sci 43(3):864-869.

19. Hato T, Tabata M, & Oike Y (2008) The role of angiopoietin-like proteins in angiogenesis and metabolism. Trends Cardiovasc Med 18(1):6-14.

20. Zhu P, Goh Y Y, Chin H F, Kersten S, & Tan N S (2012) Angiopoietin-like 4: a decade of research. Biosci Rep 32(3):211-219.

21. Le Jan S, et al. (2003) Angiopoietin-like 4 is a proangiogenic factor produced during ischemia and in conventional renal cell carcinoma. Am J Pathol 162(5):1521-1528.

22. Burkitt K, Chun S Y, Dang D T, & Dang L H (2009) Targeting both HIF-1 and HIF-2 in human colon cancer cells improves tumor response to sunitinib treatment. Mol Cancer Ther 8(5):1148-1156.

23. Knowles H J, Cleton-Jansen A M, Korsching E, & Athanasou N A (2010) Hypoxia-inducible factor regulates osteoclast-mediated bone resorption: role of angiopoietin-like 4. FASEB J 24(12):4648-4659.

24. Li H, et al. (2011) Hypoxia-inducible factor 1 alpha-activated angiopoietin-like protein 4 contributes to tumor metastasis via vascular cell adhesion molecule-1/integrin betal signaling in human hepatocellular carcinoma. Hepatology 54(3):910-919.

25. Zhang H, et al. (2012) HIF-1-dependent expression of angiopoietin-like 4 and L1CAM mediates vascular metastasis of hypoxic breast cancer cells to the lungs. Oncogene 31(14):1757-1770.

26. Kelly B D, et al. (2003) Cell type-specific regulation of angiogenic growth factor gene expression and induction of angiogenesis in nonischemic tissue by a constitutively active form of hypoxia-inducible factor 1. Circ Res 93(11):1074-1081.

27. Lam D W & LeRoith D (2012) The worldwide diabetes epidemic. Curr Opin Endocrinol Diabetes Obes 19(2):93-96.

28. Zhang X, Bao S, Hambly B D, & Gillies M C (2009) Vascular endothelial growth factor-A: a multifunctional molecular player in diabetic retinopathy. Int J Biochem Cell Biol 41(12):2368-2371.

29. Saint-Geniez M, et al. (2008) Endogenous VEGF is required for visual function: evidence for a survival role on muller cells and photoreceptors. PLoS One 3(11):e3554.

30. Ferrara N, et al. (1996) Heterozygous embryonic lethality induced by targeted inactivation of the VEGF gene. Nature 380(6573):439-442.

31. Yoshida T, et al. (2010) Digoxin inhibits retinal ischemia-induced HIF-1alpha expression and ocular neovascularization. Faseb J 24(6):1759-1767.

32. Semenza G L (2010) HIF-1: upstream and downstream of cancer metabolism. Curr Opin Genet Dev 20(1):51-56.

33. Mucaj V, Shay J E, & Simon M C (2012) Effects of hypoxia and HIFs on cancer metabolism. Int J Hematol 95(5):464-470.

34. Augustin H G, Koh G Y, Thurston G, & Alitalo K (2009) Control of vascular morphogenesis and homeostasis through the angiopoietin-Tie system. Nat Rev Mol Cell Biol 10(3):165-177.

35. Grootaert C, Van de Wiele T, Verstraete W, Bracke M, & Vanhoecke B (2012) Angiopoietin-like protein 4: health effects, modulating agents and structure-function relationships. Expert Rev Proteomics 9(2):181-199.

36. Kadomatsu T, Tabata M, & Oike Y (2011) Angiopoietin-like proteins: emerging targets for treatment of obesity and related metabolic diseases. FEBS J 278(4):559-564.

37. Ito Y, et al. (2003) Inhibition of angiogenesis and vascular leakiness by angiopoietin-related protein 4. Cancer Res 63(20):6651-6657.

38. Galaup A, et al. (2006) Angiopoietin-like 4 prevents metastasis through inhibition of vascular permeability and tumor cell motility and invasiveness. Proc Natl Acad Sci USA 103(49):18721-18726.

39. Cazes A, et al. (2006) Extracellular matrix-bound angiopoietin-like 4 inhibits endothelial cell adhesion, migration, and sprouting and alters actin cytoskeleton. Circ Res 99(11):1207-1215.

40. Yang Y H, et al. (2008) Suppression of the Raf/MEK/ERK signaling cascade and inhibition of angiogenesis by the carboxyl terminus of angiopoietin-like protein 4. Arterioscler Thromb Vasc Biol 28(5):835-840.

41. Chomel C, et al. (2009) Interaction of the coiled-coil domain with glycosaminoglycans protects angiopoietin-like 4 from proteolysis and regulates its antiangiogenic activity. FASEB J 23(3):940-949.

42. Hermann L M, et al. (2005) Angiopoietin-like-4 is a potential angiogenic mediator in arthritis. Clin Immunol 115(1):93-101.

43. Gealekman O., et al. (2008) Enhanced angiogenesis in obesity and in response to PPARgamma activators through adipocyte VEGF and ANGPTL4 production. Am J Physiol Endocrinol Metab 295(5):E1056-1064.

44. Nakayama T, et al. (2010) Expression of angiopoietin-like 4 in human gastric cancer: ANGPTL4 promotes venous invasion. Oncol Rep 24(3):599-606.

45. Nakayama T, et al. (2011) Expression of angiopoietin-like 4 (ANGPTL4) in human colorectal cancer: ANGPTL4 promotes venous invasion and distant metastasis. Oncol Rep 25(4):929-935.

46. Akishima-Fukasawa Y, et al. (2011) Histopathological predictors of regional lymph node metastasis at the invasive front in early colorectal cancer. Histopathology 59(3):470-481.

47. Padua D, et al. (2008) TGFbeta primes breast tumors for lung metastasis seeding through angiopoietin-like 4. Cell 133(1):66-77.

48. Ma T, et al. (2010) Viral G protein-coupled receptor up-regulates Angiopoietin-like 4 promoting angiogenesis and vascular permeability in Kaposi's sarcoma. Proc Natl Acad Sci USA 107(32):14363-14368.

49. Huang R L, et al. (2011) ANGPTL4 modulates vascular junction integrity by integrin signaling and disruption of intercellular VE-cadherin and claudin-5 clusters. Blood 118(14):3990-4002.

50. Ito Y, et al. (2003) Inhibition of angiogenesis and vascular leakiness by angiopoietin-related protein 4. Cancer Res 63(20):6651-6657.

51. Perdiguero E G, et al. (2011) Alteration of developmental and pathological retinal angiogenesis in angptl4-deficient mice. J Biol Chem 286(42):36841-36851.

52. Wahlin K J, et al. (2004) A method for analysis of gene expression in isolated mouse photoreceptor and Muller cells. Mol Vis 10:366-375.

53. Mori K, et al. (2002) Intraocular adenoviral vector-mediated gene transfer in proliferative retinopathies. Invest Ophthalmol Vis Sci 43(5):1610-1615.

54. Bhutto I A, et al. (2004) Localization of collagen XVIII and the endostatin portion of collagen XVIII in aged human control eyes and eyes with age-related macular degeneration. Invest Ophthalmol Vis Sci 45(5):1544-1552.

55. Montaner S, et al. (2003) Endothelial infection with KSHV genes in vivo reveals that vGPCR initiates Kaposi's sarcomagenesis and can promote the tumorigenic potential of viral latent genes. Cancer Cell 3(1):23-36.

56. Edwards M M, et al. (2011) Lamal mutations lead to vitreoretinal blood vessel formation, persistence of fetal vasculature, and epiretinal membrane formation in mice. BMC Dev Biol 11:60.

57. McLeod D S, et al. (2011) Retinal and optic nerve head pathology in Susac's syndrome. Ophthalmology 118(3):548-552.

58. McLeod D S, Hasegawa T, Prow T, Merges C, & Lutty G (2006) The initial fetal human retinal vasculature develops by vasculogenesis. Dev Dyn 235(12):3336-3347.

1. A method for treating diabetic macular edema (DME) comprising the step of administering to a subject diagnosed with DME an effective mount of an angiopoietin-like 4 protein (ANGPTL4) antagonist, wherein the effective amount treats DME in the subject.

2. The method of claim 1, wherein the ANGPTL4 antagonist is an anti-ANGPTL4 antibody.

3. The method of claim 1, further comprising the step of administering an effective amount of a vascular endothelial growth factor (VEGF) antagonist, wherein the combined effective amount treats DME in the subject.

4. The method of claim 3, wherein the VEGF antagonist is an anti-VEGF antibody.

5. The method of claim 3, wherein the administration of the ANGPTL4 antagonist and the VEGF antagonist step is performed concurrently.

6. The method of claim 3, wherein the ANGPTL4 antagonist and the VEGF antagonist are administered as a combination composition.

7. The method of claim 1, wherein the antagonist is administered intraocularly.

8. A method for treating DME comprising the step of administering to a subject diagnosed with DME an effective amount of an ANGPTL4 antagonist and an effective amount of a VEGF antagonist, wherein the combined effective amounts treat DME in the subject.

9. The method of claim 8, wherein the ANGPTL4 antagonist is an anti-ANGPTL4 antibody.

10. The method of claim 8, wherein the VEGF antagonist is an anti-VEGF antibody.

11. The method of claim 8, wherein the administration of the ANGPTL4 antagonist and the VEGF antagonist step is performed concurrently.

12. The method of claim 8, wherein the ANGPTL4 antagonist and the VEGF antagonist are administered as a combination composition.

13. A method for treating ischemic retinopathy comprising the step of administering to a subject diagnosed with ischemic retinopathy an effective amount of an ANGPTL4 antagonist.

14. The method of claim 13, wherein the ischemic retinopathy is DME or neovascular age-related macular degeneration (AMD).

15. The method of claim 13, wherein the ischemic retinopathy is retinopathy of prematurity, incontinentia pigmenti, diabetic retinopathy, retinal vein occlusion, and sickle cell retinopathy.

16. A method for treating DME comprising the step of intraocularly administering to a subject diagnosed with DME an effective amount of an ANGPTL4 antibody.

17. A method for treating AMD comprising the step of intraocularly administering to a subject diagnosed with neovascular AMD an effective amount of an ANGPTL4 antibody.

18. A method for treating DME comprising the step of intraocularly administering to a subject diagnosed with DME an effective amount of an ANGPTL4 antibody and an effective amount of a VEGF antibody.

19. A method for inhibiting ANGPTL4 in a patient diagnosed with ischemic retinopathy comprising the step of administering an effective amount of an ANGPTL4 antagonist.

20. The method of claim 19, wherein the ischemic retinopathy is DME, proliferative diabetic retinopathy (PDR) or neovascular AMD.

21. The method of claim 20, wherein the ischemic retinopathy is one or more conditions selected from the group consisting of retinopathy of prematurity, incontinentia pigmenti, diabetic retinopathy, retinal vein occlusion, sickle cell retinopathy, neovascular glaucoma, corneal neovascularization, pterygia, and pinguecula, vein occlusions including diabetic macular edema (DME), vein occlusions and retinal and choroidal neovascular diseases.


SEQUENCE LISTING (12 sequences)

SEQ ID NO: 1 - DNA, 20 bp, Homo sapiens
  misc_feature (1)..(20): Human VEGF forward primer
gggcagaatcatcacgaagt

SEQ ID NO: 2 - DNA, 20 bp, Homo sapiens
  misc_feature (1)..(20): Human VEGF reverse primer
tggtgatgttggactcctca

SEQ ID NO: 3 - DNA, 19 bp, Homo sapiens
  misc_feature (1)..(19): Human ANGPTL4 forward primer
ggacacggcctatagcctg

SEQ ID NO: 4 - DNA, 20 bp, Homo sapiens
  misc_feature (1)..(20): Human ANGPTL4 reverse primer
ctcttggcgcagttcttgtc

SEQ ID NO: 5 - DNA, 20 bp, Homo sapiens
  misc_feature (1)..(20): Human Beta actin forward primer
ctcttccagccttccttcct

SEQ ID NO: 6 - DNA, 20 bp, Homo sapiens
  misc_feature (1)..(20): Human Beta actin reverse primer
agcactgtgttggcgtacag

SEQ ID NO: 7 - DNA, 19 bp, Mus musculus
  misc_feature (1)..(19): Murine VEGF forward primer
ttactgctgtacctccacc

SEQ ID NO: 8 - DNA, 19 bp, Mus musculus
  misc_feature (1)..(19): Murine VEGF reverse primer
acaggacggcttgaagatg

SEQ ID NO: 9 - DNA, 20 bp, Mus musculus
  misc_feature (1)..(20): Murine ANGPTL4 forward primer
ttggtacctgtagccattcc

SEQ ID NO: 10 - DNA, 20 bp, Mus musculus
  misc_feature (1)..(20): Murine ANGPTL4 reverse primer
gaggctaagaggctgctgta

SEQ ID NO: 11 - DNA, 20 bp, Mus musculus
  misc_feature (1)..(20): Murine cyclophilin A forward primer
agcatacaggtcctggcatc

SEQ ID NO: 12 - DNA, 20 bp, Mus musculus
  misc_feature (1)..(20): Murine cyclophilin A reverse primer
ttcaccttcccaaagaccac